(19) United States
(12) Patent Application Publication
(10) Pub. No.: US 2023/0285635 A1
(43) Pub. Date: Sep. 14, 2023

(54) METHOD FOR PRODUCING HIGHLY FUNCTIONAL ARTIFICIAL ORGANS USING APTAMERS

(71) Applicant: KANGSTEM BIOTECH CO., LTD., Seoul (KR)

(72) Inventors: Kyung-Sun KANG, Seoul (KR); Da-Hyun KIM, Hanam-si, Gyeonggi-do (KR)

(73) Assignee: KANGSTEM BIOTECH CO., LTD., Seoul (KR)

(21) Appl. No.: 18/040,448

(22) PCT Filed: Jul. 27, 2021
(86) PCT No.: PCT/KR2021/009732

§ 371 Date: Feb. 3, 2023

(30) Foreign Application Priority Data
Aug. 6, 2020 (KR) 10-2020-0098314
Nov. 12, 2020 (KR) 10-2020-0150913

(51) Int. Cl. A61L 27/36; A61L 27/50

(52) U.S. Cl. CPC A61L 27/3687; A61L 27/507; A61L 27/3604; A61L 2430/28

(57) ABSTRACT

Provided is a method for producing highly functional artificial organs, including blood vessels capable of carrying perfusate, by using aptamers. The aptamers, according to the presently claimed subject matter, enhance blood vessel adhesion ability, viability, angiogenesis potential, and the like when used as a coating agent of a decellularized scaffold, and thus can more efficiently reconstruct vasculature than existing antibodies. Therefore, a vascularized artificial liver produced using the aptamers exhibits the effects of reducing thrombogenesis in vivo as well as enhancing liver function, and thus is expected to be applied as a method for treating diseases by using artificial organs produced using the aptamers according to the presently claimed subject matter.

### TECHNICAL FIELD

The present invention relates to a method for producing highly functional artificial organs using aptamers, and the like.

The present application claims priority to and the benefit of Korean Patent Application Nos. 10-2020-0098314 and 10-2020-0150913 filed in the Korean Intellectual Property Office on Aug. 6, 2020 and Nov. 12, 2020, respectively, and all the contents disclosed in the specification and drawings of those applications are incorporated in the present application.

### BACKGROUND ART

Artificial organ biotechnology is technology for producing devices which replace organs constituting the body by various methods, including a technique for culturing stem cells and cell growth factors (cell fluid nutrients, and the like) on a three-dimensional bio-artificial scaffold and creating an artificial organ using this formwork. Although donor organ transplantation is performed for almost all organs due to the development of modem medicine, many patients cannot benefit due to the lack of donors, and in the case of artificial organs, side effects occur due to the lack of biocompatibility.

Meanwhile, chronic liver diseases such as cirrhosis are diseases that kill about 2 million patients each year. In recent decades, liver transplantation has been conducted in many cases of end-stage liver disease due to breakthrough developments in immunology and organ transplantation, and liver transplantation has been currently recognized as the only treatment for congenital or acquired liver disease, but the number of donors is insufficient. In this respect, the production of an artificial liver through tissue engineering has been attracting attention as a substitute for donor organs. Since Korea has a very high mortality rate due to liver-related diseases compared to other diseases, has a considerable number of people waiting for transplantation due to the shortage of organs for transplantation, and is one of the countries with the highest number of liver transplants, there is a need for developing a technique capable of ameliorating these situations.

Although various methods such as tissue engineering, application of 3D printer technology, and organ reconstruction (organoid) using stem cells have been studied in order to produce a liver scaffold, technical limitations such as the inability to mimic organ fine structures and size limitations have not been overcome. As a human organ-mimicking scaffold with safety and functionality while overcoming the above problems, a decellularized scaffold in which all cells have been removed from animal organs is being evaluated as a useful substrate. Cellular components capable of inducing immune responses are removed, and simultaneously, organ-specific microenvironments such as microstructures and biochemical signals are created in scaffolds, so that there is an advantage in that the three-dimensional organization of cells in the scaffold is facilitated when human cells are injected. Therefore, decellularized scaffolds may be used as suitable substrates for producing artificial organs, and research has been conducted to reconstruct various organs using decellularized scaffolds.

Since the liver has a histologically very complex vascular structure and is an organ through which 25% or more of the cardiac output of blood passes, the vascularization of the organ can be the key to successful reconstruction of an artificial liver. After transplantation, artificial organs in which vascular structures have not been formed are connected to the recipient's bloodstream, and then, intravascular thrombi are formed due to acute immune responses, and there is a high possibility of subsequent graft failure. Therefore, various coating agents for efficiently reconstructing vascular structures have been studied.

Aptamers are single-stranded nucleic acids consisting of short sequences, and are applied as substitutes for antibodies because there are advantages of their high affinity to specific proteins, low immunogenicity, and ability to be mass-produced.

Aptamers are usually used for medical diagnosis, and are also attracting attention as therapeutic agents that target cancer or viral diseases. However, the usefulness of aptamers as coating agents in terms of tissue engineering has not been clarified.

Further, a human leukocyte antigen (HLA) is one of the human histocompatibility antigens, and immune rejection may occur in the case where homologous cells or artificial organs do not match the human leukocyte antigen type when transplanted into a patient. Recently, many studies have been conducted on universal cells established by removing such human leukocyte antigen-A, B, and C types through gene editing. However, there is a limitation that cells from which human leukocyte antigens-A, B, and C have been removed still show sensitivity to NK cells during transplantation. Therefore, cells capable of ultimately evading immune responses can be produced by overexpressing the “Don't eat me” signal, which suppresses NK cell responses. In particular, cells differentiated into various lineages from off-the-shelf universal stem cells (USCs) having such characteristics may be applied as cell compositions to minimize the occurrence of immune rejection during artificial organ production.

Therefore, in this technique, the efficiency of vasculature reconstruction was maximized using a nucleic acid aptamer as a coating agent, and a technique capable of minimizing thrombus formation after artificial organ transplantation has been established for the first time. In addition, through this technique, a vascularized artificial liver that can be transplanted in vivo using universal stem cell (USC)-derived liver-constituting cells, and the like and has enhanced functionality was produced.

### RELATED ART DOCUMENTS

K. H. Hussein, K. M. Park, K. S. Kang, H. M. Woo, Heparin-gelatin mixture improves vascular reconstruction efficiency and hepatic function in bioengineered livers, Acta Biomater 38(2016) 82-93

### DISCLOSURE

### Technical Problem

Thus, the present inventors maximized the efficiency of vasculature reconstruction using a nucleic acid aptamer as a coating agent, and have established a technique capable of minimizing thrombus formation after artificial organ transplantation for the first time. In addition, the present inventors confirmed for the first time that an artificial organ that can be transplanted in vivo using a nucleic acid aptamer and has enhanced functionality can be produced, thereby completing the present invention.

Therefore, an object of the present invention is to provide a composition for producing artificial organs, comprising aptamers.

Another object of the present invention is to provide a composition for coating blood vessels, comprising aptamers.

Still another object of the present invention is to provide a method for producing artificial organs using aptamers.

Yet another object of the present invention is to provide an artificial organ produced by the method.

However, technical problems to be solved by the present invention are not limited to the aforementioned problems, and other problems that are not mentioned may be clearly understood by those skilled in the art from the following description.

### Technical Solution

To achieve the objects of the present invention, the present invention provides a composition for producing artificial organs, comprising aptamers.

Furthermore, the present invention provides a use of a composition comprising aptamers for producing artificial organs.

Further, the present invention provides a method for producing artificial organs, the method comprising a step of treating with aptamers.

In addition, the present invention provides a composition for coating blood vessels, comprising aptamers.

Furthermore, the present invention provides a use of a composition comprising aptamers for coating blood vessels.

Further, the present invention provides a method for coating blood vessels, the method comprising a step of treating with a composition comprising aptamers.

In addition, the present invention provides an artificial organ produced by the method.

In an exemplary embodiment of the present invention, the artificial organ may be biocompatible.

In another exemplary embodiment of the present invention, the artificial organ is not limited as long as it is an organ comprising blood vessels, and may be specifically a liver, but is not limited thereto.

In still another exemplary embodiment of the present invention, the aptamer may be an anti-CD31 aptamer, but is not limited thereto.

In yet another exemplary embodiment of the present invention, the aptamer may comprise a base sequence represented by SEQ ID NO: 1, but is not limited thereto.

In yet another exemplary embodiment of the present invention, the aptamer may be characterized by being specific for vascular endothelial cells, but is not limited thereto.

In yet another exemplary embodiment of the present invention, the aptamer may increase the expression of one or more selected from the group consisting of integrin beta 3 and phosphorylated Akt, but is not limited thereto.

In yet another exemplary embodiment of the present invention, the aptamer may decrease the expression of cleaved caspase-3, but is not limited thereto.

In yet another exemplary embodiment of the present invention, the composition may further comprise one or more cells selected from the group consisting of parenchymal cells and non-parenchymal cells, but is not limited thereto.

In yet another exemplary embodiment of the present invention, the cells may be stem cell-derived cells, but are not limited thereto.

In yet another exemplary embodiment of the present invention, the cells may be characterized by comprising stem cell-derived differentiated cells, but are not limited thereto.

In yet another exemplary embodiment of the present invention, the composition may further comprise stem cell-derived parenchymal cells, but is not limited thereto.

In yet another exemplary embodiment of the present invention, the composition may further comprise stem cell-derived non-parenchymal cells, but is not limited thereto.

In yet another exemplary embodiment of the present invention, the stem cells may be one or more selected from the group consisting of induced pluripotent stem cells (iPSCs), embryonic stem cells, mesenchymal stromal cells (MSCs), off-the-shelf universal stem cells (USCs), marrow-derived stem cells, adipose tissue-derived stem cells and placenta-derived stem cells, but are not limited thereto.

In yet another exemplary embodiment of the present invention, the off-the-shelf universal stem cells may have one or more of the following characteristics, but are not limited thereto.

the HLA gene has been removed; and

“Don't eat me” signals are overexpressed.

In yet another exemplary embodiment of the present invention, the preparation method may comprise the following steps, but is not limited thereto.
a) providing an organ from a tissue source;
b) decellularizing the provided organ; and
c) treating the decellularized organ with aptamers.

In yet another exemplary embodiment of the present invention, the preparation method may further comprise one or more steps selected from the group consisting of the following steps, but is not limited thereto.
d-1) recellularizing a decellularized organ into vascular endothelial cells;
d-2) recellularizing a decellularized organ into parenchymal cells; and
d-3) recellularizing a decellularized organ into non-parenchymal cells.

### Advantageous Effects

When the aptamer of the present invention is used as a coating agent of a decellularized scaffold, it is possible to more efficiently reconstruct vasculature than existing antibodies by enhancing blood vessel adhesion ability, viability, angiogenesis potential, and the like. Therefore, a vascularized artificial liver produced using the aptamers exhibits the effects of reducing thrombogenesis in vivo as well as enhancing liver function, and thus is expected to be applied as a method for treating diseases by using artificial organs produced using the aptamers according to the present invention.

### DESCRIPTION OF DRAWINGS

FIGS. 1A to 1G illustrate the results of analyzing the characteristics of anti-CD31 aptamers: FIG. 1A illustrates the functional structure of an anti-CD31 aptamer that forms a CD31-aptamer complex; FIGS. 1B to 1D illustrate the results of confirming the dose-dependent binding of a cy5-labeled anti-CD31 aptamer to HUVECs, HepG2 cells and MSCs, FIG. 1B illustrates the dose-dependent binding efficiency of an anti-CD31 aptamer with a FACS profile of HUVECs, HepG2 cells and MSCs, FIG. 1C illustrates the results of quantifying the binding rate, and FIG. 1D illustrates the results of quantifying the mean fluorescence intensity (MFI) of anti-CD31 aptamers bound to HUVECs, HepG2 cells and MSCs; FIG. 1E illustrates immunocytochemical images of HUVECs stained with a cy5-labeled anti-CD31 aptamer (top, red) and an anti-CD31 antibody (bottom, red); and FIGS. 1F and 1G illustrate immunocytochemical images of HepG2 cells and MSCs stained with an anti-CD31 aptamer (top, red) and an anti-CD31 antibody (bottom, red).

FIGS. 2A to 2J show that an anti-CD31 aptamer enables integrin-mediated adhesion of endothelial cells (ECs) to an extracellular matrix (ECM) under shear stress: FIG. 2A illustrates ECM components; FIG. 2B illustrates representative phase-contrast images of sheared HUVECs; FIG. 2C illustrates the attached rate; FIG. 2D illustrates the results of qRT-PCR analysis of coating a device with PBS (vehicle, uncoated), an anti-CD31 aptamer (APT-coated) and an anti-CD31 antibody (Ab-coated), respectively; FIGS. 2E to 2G illustrate western blot analysis results of integrin beta 3, total Akt and phosphorylated Akt in static HUVECs and exposed HUVECs after shear stress exposure in microfluidic devices coated with the respective coating agents; FIGS. 2H to 2J illustrate western blot analysis results of cleaved caspase-3 expression in HUVECs exposed to shear stress and static HUVECs in groups coated with PBS (vehicle, uncoated), an anti-CD31 aptamer (APT-coated) and an anti-CD31 antibody (Ab-coated), respectively; and FIG. 2J illustrates the qRT-PCR results of nitric oxide synthase 3 (NOS3) expression in sheared HUVECs of each group normalized to static HUVECs.

FIGS. 3A to 3L illustrate the results of efficient re-endothelialization using HUVECs of decellularized liver scaffolds: FIG. 3A illustrates a method for re-endothelialization of decellularized rat liver scaffolds using HUVECs; FIG. 3B illustrates representative immunofluorescence images of endothelial vessels with CFDA-labeled HUVECs on scaffolds coated or uncoated with an anti-CD31 aptamer (APT-coated) or an anti-CD31 antibody (Ab-coated); FIG. 3C illustrates the endothelial coverage of the re-endothelialized vessels of each scaffold; FIG. 3D quantifies the average number of re-endothelialized blood vessels of each scaffold; FIG. 3E illustrates the results of quantifying intravascular dextran discharged from the inferior vena cava in each construct after perfusion of dextran through the portal vein; FIG. 3F illustrates enlarged confocal images of APT-coated constructs stained with zonula occludens-1 (ZO-1); FIG. 3G illustrates the qRT-PCR detection results of VE-cadherin and Claudin 5 (CLDN5) in static HUVECs, uncoated, APT-coated and Ab-coated re-endothelialized constructs; FIG. 3H illustrates the results of confirming the viability of each construct using PrestoBlue reagent on days 3, 5, and 7 by performing a resazurin reduction perfusion assay; FIG. 3I illustrates representative confocal images of re-endothelialized constructs of each group stained with cleaved caspase-3 (red) and DAPI (blue).; FIG. 3J illustrates the results of quantifying cells expressing cleaved caspase-3 in each group; FIG. 3K illustrates the amount of NO produced as quantified by ELISA in re-endothelialized constructs according to each group; and FIG. 3L illustrates the results of ELISA quantification of human VEGF secreted from re-endothelialized constructs in each group on day 7.

FIGS. 4A to 4G are results showing that re-endothelialized constructs form low thrombi after human blood perfusion: FIG. 4A illustrates the method used to transfer human blood to re-endothelialized structures in order to evaluate thrombus formation; FIG. 4B illustrates the macroscopic image of each scaffold after human blood perfusion, in which non-endothelialized DLM was used as a negative control; FIG. 4C illustrates representative immunohistochemical images of harvested constructs from each group stained with integrin αIIb (green); FIG. 4D illustrates the quantification of the fluorescence intensity of integrin αIIb expression; FIG. 4E illustrates the results of performing platelet quantification using a hematology analyzer after the blood perfusate in the DLM group was harvested at certain time points; FIG. 4F illustrates the results of performing platelet quantification using a hematology analyzer after blood perfusate was harvested from re-endothelialized constructs of each group at certain time points; and FIG. 4G illustrates the results of RT-PCR analysis of CD63, PLSCR1, TBXAS1 and THBS1, which are genes involved in platelet aggregation in blood perfusion constructs of each group.

FIGS. 5A to 5M illustrate the results of confirming VBHL structures with enhanced functional maturation: FIG. 5A is a schematic view describing the recellularization process of decellularized rat liver scaffolds using HepG2 cells, LX2 cells, HUVECs and MSCs; FIG. 5B is a set of representative immunofluorescence images of an uncoated VBHL construct (CTL-VBHL) or VBHL construct coated with an anti-CD31 aptamer (APT-VBHL) and an anti-CD31 antibody (Ab-VBHL) on day 21; FIG. 5C illustrates the endothelial coverage of blood vessels of each group; FIG. 5D illustrates the results of quantifying the number of average endothelialized vessels per field; FIG. 5E is a set of representative immunohistochemical images of CTL-VBHL, APT-VBHL and Ab-VBHL constructs stained with α-SMA (red); FIG. 5F illustrates the dextran perfusion analysis results; FIGS. 5G to 5H illustrate the results of ELISA quantification of VEGF and NO secretion from VBHL constructs in each group at the indicated time points; FIGS. 5I and 5J illustrate the results of ELISA quantification of the amounts of albumin and urea secreted from VBHL constructs in each group at the indicated time points; FIG. 5K shows the results illustrating improved viability in APT-VBHL constructs on days 17 and 20; FIG. 5L illustrates representative confocal images of TUNEL assay; and FIG. 5M illustrates the results of quantifying TdT-positive cells in a randomized field.

FIGS. 6A to 6E illustrate successful in vivo reperfusion results of anti-CD31 aptamer-coated VBHL constructs: FIG. 6A illustrates auxiliary grafting photographs of the VBHL construct, in which the renal vein (yellow arrow) was connected to the inferior vena cava of the VBHL construct, the renal artery (white arrow) was connected to the portal vein of the construct, and the VBHL construct was reperfused with the in vivo renal circulation system after removal of the vascular clamp (blue arrow); FIG. 6B illustrates representative images of the construct harvested after transplantation; FIG. 6C is a set of representative confocal images of the harvested constructs of each group stained with integrin αIIb (green); FIG. 6D illustrates the results of quantifying the fluorescence intensity of integrin αIIb expression; and FIG. 6E illustrates the results of RT-PCR analysis of Cd63, Plscr1 and Thbs1 in each VBHL construct after transplantation.

FIGS. 7A to 7F illustrate the results of transplanting VBHL constructs in thioacetamide (TAA)-induced liver fibrosis: FIG. 7A is a schematic view of the method of transplanting VBHL constructs into TAA-induced liver cirrhosis rats; FIG. 7B illustrates representative H&E staining images of host liver tissue sections and representative picrosirius red staining results of liver sections harvested on week 4 after transplantation of each construct; FIG. 7C illustrates the results of quantifying the degree of fibrosis in host livers of each group; FIG. 7D illustrates the qRT-PCR analysis results of fibrosis-related genes, αSma, Vimentin, Tgf-beta1 and Timp1, represented in host livers of each group; and FIGS. 7E and 7F illustrate the ELISA measurement results of ALT and AST levels in rat serum, in which the red line means the normal range.

### MODES OF THE INVENTION

Hereinafter, the present invention will be described in detail.

The present invention provides a composition for producing artificial organs, comprising aptamers.

Furthermore, the present invention relates to a composition for coating blood vessels, comprising aptamers.

Tissue engineering is a method of anchoring cells on a certain construct (template) using a combination of cells and various materials, and such a construct (template) is called a scaffold, and the purpose of making this is to create a construct capable of replacing a damaged tissue or organ, that is, an organ. Although there are various types, a construct (template) that leaves only outlines of the microstructure and the organ after cells are removed by decellularizing a biological construct (that is, organ) called a bioscaffold.

The artificial organ of the present invention may be characterized in that a bioscaffold is treated with aptamers to reconstruct a target organ structure, such as a vascular structure, but is not limited thereto. Specifically, the aptamer of the present invention may reconstruct or produce a vascular structure by coating the vascular wall of a decellularized scaffold to specifically bind to vascular endothelial cells.

The present inventors confirmed that when the vascular wall of a decellularized scaffold was coated with an anti-CD31 aptamer such that vascular endothelial cells could adhere to the wall of the decellularized scaffold, a highly functional vasculature, in which the function of the vascular barrier was maintained, was reconstructed (see the Examples of the present invention).

In the present invention, the blood vessel may be the blood vessel of a decellularized scaffold, but is not limited thereto.

In the present invention, the blood vessel may be a hepatic vessel, for example, the hepatic portal vein, hepatic sinusoid, a hepatic vein or the hepatic artery, but is not limited thereto.

In the present invention, the artificial organ may be biocompatible. The term biocompatibility in the present invention is used interchangeably with “transplant compatibility” and refers to a property of not causing immune rejection upon cell transplantation. A cell, tissue, or organ may be rendered transplant compatible by reducing the expression of genes responsible for immune rejection, and as a non-limiting example thereof, reducing the expression level of the HLA gene may render a cell, tissue or organ transplant compatible. Transplant compatible cells comprise cells in which immunocompatible antigens are homozygous or null, but are not limited thereto.

In the present invention, the artificial organ may be, for example, a liver, kidney, heart, valve, ureter, urinary bladder, lung, pancreas, and the like, but is not limited thereto.

In the present invention, the artificial organ may be an organ comprising blood vessels, but is not limited thereto.

In the present invention, an aptamer refers to a single-stranded nucleic acid (DNA, RNA, or modified nucleic acid) having a stable tertiary structure by itself as a material capable of specifically binding to a target material, and can bind to a target protein (material). The aptamer may be prepared by determining a sequence of an oligonucleotide which is selective and has binding ability to a target material to be confirmed and synthesizing the sequence according to a general method of preparing an aptamer, and then modifying the 5′ end or 3′ end of the oligonucleotide with —SH, —COOH, —OH or NH₂, such that the end can bind to a functional group of an aptamer chip, but is not limited thereto.

In the present invention, the aptamer may be an anti-CD31 aptamer, but is not limited thereto.

In the present invention, the anti-CD31 aptamer may comprise a base sequence represented by SEQ ID NO: 1, or may consist of the base sequence represented by SEQ ID NO: 1, but is not limited thereto.

In the present invention, the aptamer may be characterized by being specific for vascular endothelial cells, but is not limited thereto. In addition, the aptamer of the present invention may be used not only for vascular reconstruction, but also for hepatic parenchymal reconstruction, bile duct reconstruction, and the like, regardless of the type of organ during organ reconstruction by targeting only vascular endothelial cells. That is, aptamers that bind to proteins specifically expressed by hepatic parenchymal cells may be used during hepatic parenchymal reconstruction, and aptamers that bind to proteins specifically expressed by cholangiocytes may be used during bile duct reconstruction.

Therefore, the aptamer of the present invention may be specific for a protein specifically expressed by target cells, but is not limited thereto.

In the present invention, the aptamer may increase the expression of one or more selected from the group consisting of integrin beta 3 and phosphorylated Akt, but is not limited thereto.

In the present invention, the aptamer may decrease the expression of cleaved caspase-3, but is not limited thereto.

In the present invention, the composition may typically be delivered to a tissue or organ matrix by a cell-miscible solution (for example, a physiological composition) under physiological conditions (for example, 37° C.). The composition may include a buffer, a nutrient (for example, a sugar and carbohydrate), an enzyme, a proliferation and/or differentiation medium, a cytokine, an antibody, an inhibitory factor, a growth factor, a salt solution, or a serum-derived protein, and the like, but is not limited thereto.

In the present invention, the composition may further comprise parenchymal cells, but is not limited thereto. As used herein, the term “parenchymal cells” refers to cells that form the main part that performs functions inherent to cells. In the present invention, the parenchymal cells may specifically be hepatic parenchymal cells, renal parenchymal cells, corneal parenchymal cells, vascular endothelial cells, and the like, preferably hepatic parenchymal cells, that is, hepatocytes, but are limited thereto. In the present invention, hepatic parenchymal (parenchymal hepatocyte) cells make up a total of 80% of the liver under non-pathological conditions.

In the present invention, the parenchymal cells may be derived from the same species as a recipient, but are not limited thereto.

In the present invention, the composition may further comprise stem cell-derived parenchymal cells, but is not limited thereto. The parenchymal cells may be parenchymal cells differentiated from human off-the-shelf universal stem cells from which human leukocyte antigens have been removed and/or which overexpress “Don't eat me” signals, but are not limited thereto.

In the present invention, the composition may further comprise non-parenchymal cells, but is not limited thereto. The non-parenchymal cells may be one or more selected from the group consisting of hepatic endothelial cells, Kupffer cells, hepatic stellate cells, Ito cells, lipocytes, fat-storing cells, cholangiocytes, pit cells, vascular epithelial cells and fibroblasts, but are not limited thereto.

In the present invention, the composition may further comprise stem cell-derived non-parenchymal cells, but is not limited thereto. The stem cell-derived non-parenchymal cells may be non-parenchymal cells differentiated from human off-the-shelf universal stem cells from which human leukocyte antigens have been removed and/or which overexpress “Don't eat me” signals, but are not limited thereto.

In the present invention, the cells may be characterized by comprising stem cell-derived differentiated cells, but are not limited thereto.

As used herein, the term “stem cells” refers to cells which have the property capable of continuously producing the same cells as themselves for a certain period of time in a differentiated state and the property of differentiating into specific cells under suitable conditions.

In the present invention, the stem cells may be one or more selected from the group consisting of induced pluripotent stem cells (iPSCs), embryonic stem cells, mesenchymal stromal cells (MSCs), off-the-shelf universal stem cells (USCs), marrow-derived stem cells, adipose tissue-derived stem cells and placenta-derived stem cells, but are not limited thereto.

In the present invention, the off-the-shelf universal stem cells may be characterized in that HLAs have been removed, or “Don't eat me” signals are overexpressed, but are not limited thereto. The present invention is expected to be clinically applicable by constructing a universal artificial organ comprising stem cell-derived constitutive cells from which human leukocyte antigens have been removed and/or which overexpress “Don't eat me” signals to minimize immune rejection during transplantation.

In the present invention, the off-the-shelf universal stem cells may be HLA class I knockout iPSC cells or HLA class I knockout MSC cells, but are not limited thereto.

In the present invention, the “Don't eat me” signal may be CD47 or CD24, but is not limited thereto.

Further, the present invention provides a method for producing artificial organs, the method comprising a step of treating with aptamers.

In the present invention, the method for producing artificial organs may comprise the following steps, but is not limited thereto:
a) providing an organ from a tissue source;
b) decellularizing the provided organ; and
c) treating the decellularized organ with aptamers.

In the present invention, the method may further comprise one or more steps selected from the group consisting of d-1) recellularizing the decellularized organ into vascular endothelial cells;
d-2) recellularizing the decellularized organ into parenchymal cells; and
d-3) recellularizing the decellularized organ into non-parenchymal cells, but is not limited thereto.

In the present invention, the tissue source may be selected from various mammalian groups such as humans, monkeys, mice, rats, pigs, cows and rabbits.

In the present invention, the decellularization may be performed by methods known in the art, but in an exemplary embodiment of the present invention, the hepatic portal vein obtained from the tissue source is washed with deionized water and PBS, then washed with deoxyribonuclease, and then sterilized with peroxyacetic acid. For example, the following references describe the perfusion-based decellularization of the lungs, liver, kidneys, brain and limbs: Van Putte et al., 2002, Ann. Thorac. Surg., 74(3):893-8; den Butter et al., 1995, Transpl. Int., 8:466-71; Firth et al., 1989, Clin. Sci. (Lond.), 77(6):657-61; Mazzetti et al., 2004, Brain Res., 999(1):81-90; Wagner et al., 2003, J. Artif Organs, 6(3):183-91. As an alternative to perfusion-based decellularization, biological tissues and organs may be decellularized by immersion in a decellularization solution that removes cells. See, for example, U.S. Pat. Nos. 6,376,244 and 6,753,181.

In the present invention, physiological buffers suitable for perfusion comprise nutrient supplies that may be used for storage and/or organ perfusion, comprising transplantation, and examples comprise buffers with glucose, EGM-2, EGM-2MV, DMEM, Promocell endothelial cell medium, Medium 200, and DMEMF/12, but are not limited thereto. Further, the buffer comprises a culture medium solution suitable for endothelial cell culture or phosphate buffered saline (PBS), but is not limited thereto.

In the present invention, alternating the direction of perfusion (for example, anterograde and retrograde) during decellularization may help effectively remove cells from the whole organ or tissue. The decellularization as described in the present specification may cause little damage to the ECM by essentially removing cells from within the organ with little damage to the ECM, but is not limited thereto. Organs or tissues may be decellularized at a suitable temperature between 4 to 40° C. Depending on the size and weight of the organ or tissue, the specific detergent(s) in a cell disintegration medium, and the concentration of the detergent(s), the organ or tissue is generally perfused with the cell disintegration medium for about 2 to about 12 hours per gram of solid organ or tissue. Organs comprising lavage fluid may be perfused for about 1 to about 12 hours per gram of tissue. Perfusion is generally regulated by physiological conditions comprising pulsatile blood flow, flow velocity and pressure.

In the present invention, for recellularization to generate an organ or tissue, the number of regenerative cells introduced into and onto a decellularized organ may vary depending on both the type and developmental stage of an organ (for example, what the organ is and the size and weight of the organ) or tissue and regenerative cell. Different types of cells may have different tendencies with respect to the population density that these cells reach. Similarly, different organs or tissues may be recellularized at different densities. For example, a decellularized organ or tissue may be “inoculated” with at least 1,000, 10,000, 100,000, 1,000,000, 10,000,000 or 100,000,000 regenerative cells; or may have about 1,000 cells/tissue mg (wet weight, that is, weight before decellularization) to about 100,000,000 cells/tissue mg (wet weight) after recellularization, but is not limited thereto.

In the present invention, the method may further comprise culturing an organ treated with an aptamer in a bioreactor for 1 to 100 days, 1 to 90 days, 1 to 80 days, 1 to 70 days, 1 to 60 days, 1 to 50 days, 1 to 40 days, 1 to 1 month, 1 to 3 weeks, 1 to 15 days, 1 to 2 weeks, 5 to 15 days, 5 to 2 weeks, 1 week to 3 weeks or about 2 weeks, but is not limited thereto.

In addition, the present invention provides an artificial organ produced by the method.

The artificial organ may not induce immune rejection in a recipient after transplantation as an organ for transplantation, but is not limited thereto.

The artificial organ may be used for therapeutic agent screening applications, preferably for hepatic disease therapeutic agent screening applications, but is not limited thereto.

Throughout the specification of the present application, when one part comprises one constituent element, unless otherwise specifically described, this does not mean that another constituent element is excluded, but means that another constituent element may be further comprised. Throughout the specification of the present application, a term of a degree, such as about or substantially, is used in a corresponding numerical value or used as a meaning close to the numerical value when natural manufacturing and material tolerance errors are presented in a described meaning, and is used to prevent an unscrupulous infringer from illegally using disclosed contents comprising a numerical value illustrated as being accurate or absolute in order to help understanding of the present invention. Throughout the specification of the present application, a term such as a step . . . or a step of . . . , does not mean a step for . . . .

Throughout the specification of the present application, the term combination(s) thereof comprised in the Markush type expression means a mixture or combination of at least one selected from the group consisting of constituent elements described in the Markush type expression, and means comprising at least one selected from the group consisting of the constituent elements.

Throughout the specification of the present application, the description A and/or B means A or B, or A and B.

Specific steps may be performed out of the order described in cases where certain embodiments can be implemented differently. For example, two steps described in succession may be performed substantially concurrently, or may be performed in reverse order to that described.

Terms or words used in the specification and the claims should not be interpreted as being limited to typical or dictionary meanings and should be interpreted with a meaning and a concept that are consistent with the technical spirit of the present invention based on the principle that an inventor can appropriately define the concept of a term in order to describe his/her own invention in the best way.

Hereinafter, preferred examples for helping with understanding of the present invention will be suggested. However, the following examples are provided only so that the present invention may be more easily understood, and the content of the present invention is not limited by the following examples.

Experimental Methods and Materials

1. Aptamer Preparation and Analysis

An anti-CD31 single-stranded DNA aptamer (76 mer, 21966-18-01) having a core sequence of SEQ ID NO: 1 was synthesized and characterized by Aptamer Sciences Inc. (Korea). After the anti-CD31 aptamer was dissolved in sterile Ultrapure DEPC-treated water (Invitrogen, USA), the resulting solution was stored at −20° C. Before experiments were performed, the aptamer was heated at 95° C. for 10 minutes and allowed to cool at room temperature to induce proper folding. To confirm that the anti-CD31 aptamer specifically targets CD31+ ECs, immunofluorescent staining and flow cytometry were performed using an aptamer labeled with cy5 at the 5-end (5′-cy5-aptamer-3′)

<Anti-CD31 aptamer>

(SEQ ID NO: 1; core sequence)
5′-TCA GCC GCC AGC CAG TTC(primer) -
G6A GAG GAG G6A CG6 AA6 G6C 6GG G6A 6AC
CCC GA6 AA6 6
- GAC CAG AGC ACC ACA GAG(primer)-3′

6=NapdU [5-(N-Napthylcarboxyamide)-2′-deoxyuridine]

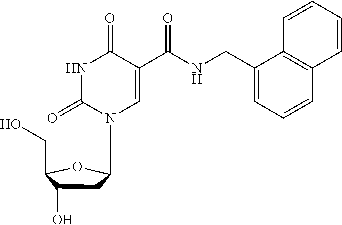

2. Cell Culture

Human umbilical vein endothelial cells (HUVECs) and human umbilical cord blood-derived mesenchymal stem cells (MSCs) purchased from ATCC (USA) were maintained in an endothelial growth medium-2 (EGM-2, Lonza, Switzerland). Under the approval of the Seoul National University Institutional Review Board (IRB No. 1608/001-021), MSCs were established as described in Donor-dependent variation of human umbilical cord blood mesenchymal stem cells in response to hypoxic preconditioning and amelioration of limb ischemia, Exp Mol Med 50(4)(2018) 35. Hepatic carcinoma cells (HepG2 cells) purchased from ATCC and LX-2 human stellate cells purchased from Millipore (USA) were cultured in high-glucose-containing Dulbecco's Modified Eagle Medium (DMEM, Hyclone, USA). All media were supplemented with 10% fetal bovine serum (FBS, Gibco), antibiotics (100 U/ml penicillin and 100 μg/ml streptomycin (1% P/S, Gibco)) and 20 μg/ml Primocin (InvivoGen, USA). The culture medium was replaced every 48 hours. Cells were maintained at 37° C. in a 5% CO₂ humidified incubator.

3. Microfluidic Device Design and Cell Detachment Analysis

A cell detachment rate according to a coating agent was evaluated using a 3D microfluidic system. A device having three microchannels was manufactured by soft lithography and replication molding using polydimethylsiloxane (PDMS, Sylgard 184; Dow Corning, USA). A PDMS prepolymer consisting of a mixture of a PDMS base and a curing agent (10:1) was poured onto a silicon wafer and cured. Subsequently, the PDMS block was fully solidified, and then separated from the wafer.

After an inlet and an outlet were punched using a biopsy punch, the PDMS block was attached to a vacuum adhesive polycarbonate film. Before additional experiments were performed, the device was kept overnight in a drying oven to restore hydrophobicity and sterilized by UV irradiation. The microfluidic device was coated with 100 μg/ml of a mixture of type 1 collagen (rat tail, #354236, Coming, USA), vitronectin (#A31804, Gibco) and fibronectin (#356008, Coming).

Thereafter, the device was coated with each of the following coating agents; PBS (negative control), 600 nM anti-CD31 aptamer and 50 μg/mL anti-CD31 antibody. 5×10⁵ HUVECs were seeded into each channel and incubated for 2 hours. Subsequently, fluid shear stress was applied to a HUVEC monolayer using an infusion syringe pump (PHD 2000 Infusion, Harvard device, USA). Shear stress was calculated based on the modified Poiseuille equation (see Equation 1).

Tw=6μQ/wH² [Equation 1]

(Tw: shear stress (dynes/cm2), μp: viscosity (Poise) at 37° C., Q: flow rate (mL/s), w: channel width (cm) and H: channel height (cm).)

EGM-2 supplemented with 1% FBS was allowed to pass through the channel at an indicated flow rate for 10 minutes. For each flow, after flow, images of cells adhered to the surface were captured using an optical microscope (IX70, Olympus, Japan) and counted using Image J software. To analyze changes in HUVECs in response to shear stress, RNA and protein were extracted from cells after exposure to a shear stress of 10 dynes/cm² for 30 minutes.

4. Production of Decellularized Rat Liver Scaffolds

Natural livers were obtained from 8-week female Sprague-Dawley rats (250 to 300 g) after systemic heparinization. A hepatic portal vein was cannulated with a 24G catheter and perfused with deionized aqueous phase 0.1% SDS (Sigma Aldrich, USA) for 6 hours and then washed with PBS for 10 hours. Subsequently, the scaffold was perfused with 0.1 mg/ml deoxyribonuclease 1 (DNase 1; Sigma Aldrich) for 1 hour and washed with PBS for 2 hours. To remove the bioburden from the scaffold, the scaffold was sterilized with 0.1% peracetic acid (Sigma Aldrich) and stored at 4° C. in PBS containing antibiotics. All rat experiments were approved by the Seoul National University Animal Care Committee (SNU-170113-2).

5. Re-Endothelialization of Rat Liver Scaffold

After EGM-2 was perfused for 1 hour to stabilize the sterile decellularized liver matrix scaffolds, the scaffolds were coated at 4° C. for 1 hour by injecting the following respective coating agents via the hepatic portal vein: PBS, 600 nM anti-CD31 aptamer and 50 μg/mL anti-CD31 antibody (14-0319-80, eBioscience, USA). A total of 1×10⁷ HUVECs were labeled with carboxyfluorescin diacetate succinimidyl ester (CFDA) using Vybrant CFDA SE cell tracker kit (Invitrogen).

CFDA-labeled HUVECs and antibiotics suspended in EGM-2 supplemented with 10% FBS were delivered through the portal vein at a rate of 0.5 ml/min. After static culture was maintained for 2 hours, the re-endothelialized construct was perfused with the medium at a rate of 1.5 ml/min using a peristaltic infusion pump in a bioreactor. The culture medium was replaced every 48 hours and samples were harvested on day 7 for additional analysis. The endothelial coverage and the number of re-endothelialized vessels were quantified by Image J software.

6. Delivery of Hepatic Parenchymal Cells and Non-Parenchymal Cells to Decellularized Rat Livers

When a decellularized liver scaffold was produced, the bile duct was cannulated with a 26G catheter and the portal vein was cannulated with a 24G catheter. After decellularization, a total of 4×10⁷ HepG2 cells were delivered through the bile duct to the parenchyma of the scaffold in half and maintained in the bioreactor for 2 days. Two days later, 4×10⁷ HepG2 cells were substantially delivered again and the construct was cultured for up to 14 days.

On day 14, a mixture of 6×10⁶ CFDA-labeled HUVECs and 3×10⁶ MSCs was delivered through the portal vein to the vessel lumen and simultaneously, 1×10⁷ LX2 cells were injected through the bile duct. All cells were delivered at a rate of 0.5 ml/min. After the static culture was maintained for 2 hours, the construct was perfused at a rate of 1.5 ml/min using a peristaltic infusion pump. Up to 14 days, the VBHL construct was maintained in a DMEM high glucose medium comprising 10% FBS and antibiotics. From day 14 to day 21, the medium was replaced with EGM-2 comprising 10% FBS and antibiotics. Additional analysis was performed on the VBHL construct obtained on day 21.

7. Statistical Analysis

Statistical analysis was performed using GraphPad Prism version 5.0. All values were reported using mean+ standard deviation. A value of p<0.05 was considered significant. Statistical analysis between both groups was performed with an unpaired, two-tailed Student's t-test. For multiple group comparisons, a two-way ANOVA was performed with the Bonferroni post hoc test. The presented data represents the results of at least three independent experiments.

8. Immunofluorescence Staining

Cells or tissue sections were fixed with 4% formaldehyde for 10 minutes. Subsequently, samples were permeabilized with 0.2% Triton X-100 (Sigma Aldrich) for 10 minutes and blocked with 5% chlorine serum (Vector Laboratories, Switzerland) for 1 hour. Subsequently, samples were probed with the following primary antibodies at 4° C. overnight: anti-human CD31 (14-0319-80, eBioscience, USA), anti-Albumin (GTX102419, GeneTex, USA), anti-Vimentin (ab45939, Abcam, UK), anti-Galactosidase alpha (GTX101178, GeneTex), anti-ZO-1 (#40-2200, Invitrogen), anti-cleaved Caspase-3 (#9664, Cell Signaling Technology, USA), anti-phospho-Akt (#9271, Cell Signaling Technology), anti-Integrin beta 3 (#13166, Cell Signaling Technology), anti-Integrin αIIb (sc-365938, Santa Cruz biotechnology, USA) and anti-α-Smooth muscle actin (ab7814, Abcam). Fluorescent-dye conjugated secondary antibodies (Alexa Fluor 488-labeled and 549-labeled; Invitrogen) were applied for 1 hour. Nuclei were stained with DAPI (sc3598, Santa Cruz Biotechnology) for 10 minutes and cells were mounted on a fluorescent mounting medium (S302380, DAKO, Denmark). Stained signals were visualized by an Eclipse TE 2000 confocal laser scanning microscope (Nikon, Japan). Similarly, paraffin-embedded tissue sections were deparaffinized, hydrated, fixed with 4% formaldehyde, and then stained as described above.

9. Flow Cytometry

To estimate the binding kinetics of anti-CD31 aptamers in a concentration-dependent manner, HUVECs were cultured with different concentrations of a cy5-labeled anti-CD31 aptamer in a reaction buffer consisting of 1 mM MgCl₂ and 2 mg/ml bovine serum albumin at 4° C. for 20 minutes. HepG2 cells and MSCs were compared in order to test the specificity of the anti-CD31 aptamer. Cells were washed with 1 ml of reaction buffer, suspended in PBS and analyzed by FACS Calibur (BD Bioscience, USA). A PE-conjugated anti-CD31 antibody (BD555446, BD Biosciences) was used as a positive control. Data was analyzed using FlowJo software (USA).

10. Cell Adhesion Assay

Rat abdominal aortas were harvested and immersed in stirred 0.05% SDS for decellularization. An internal structure was revealed by cutting the decellularized arterial scaffold longitudinally, and sterilized with 0.1% peroxyacetic acid. Subsequently, the scaffold was immersed in the coating agent for 2 hours; PBS (negative control), 600 nM anti-CD31 aptamer and 50 μg/mL anti-human CD31 antibody (14-0319-80, eBioscience, positive control). Subsequently, the coated scaffold was transferred to a 24-well plate and 1×10⁶ HUVECs were seeded onto the inner surface of the arterial scaffold. After static culture was maintained for the indicated time points (2 and 4 hours), the cell-dispensed scaffold was washed with PBS and transferred to a new plate. The scaffold was subjected to a tetrazolium-based MTT assay to quantify the number of viable cells. For the MTT assay, samples were kept in a medium containing a 10% MTT stock solution (Sigma Aldrich) at 37° C. for 4 hours. Subsequently, the supernatant was removed and dimethyl sulfoxide was added to each well for solubilization of purple formazan. Absorbance at a wavelength of 540 nm was measured using a microplate reader (Infinite M200 pro, Tecan, Switzerland).

11. qRT-PCR

Total RNA was extracted from cells or engineered constructs using NucleoZOL (Macherey-Nagel, Germany). Thereafter, complementary DNA was synthesized from the extracted RNA using the Superscript III First-Strand Synthesis System (Invitrogen). qRT-PCR was performed with SYBR Green PCR Master Mix (Applied Biosystems, USA) using the ABI 7300 Real time PCR system (Applied Biosystems). The relative quantification of target mRNA expression levels was determined by the 2(ΔΔ^{threshold cycle})(2-^{ΔΔCT}) method. Expression levels for each gene were normalized to housekeeping gene expression. The primer sequences used are shown in Table 1.

TABLE 1

| | | 5-Forward | 5-Reverse |
|---|---|---|---|
| Species | Genes | sequence-3 | sequence-3 |
| human | GAPDH | TGATGACATCAAGAA | ACCCTGTTGCTGTAG |
| | | GGTGGTG | CCAAAT |
| human | ITGB1 | CGTAGCAAAGGAACA | GGTAGTAGAGGTCAA |
| | | GCAGAG | TGGGATAGTC |
| human | ITGB3 | CCTCATCACCATCCA | GTTGTTGGCTGTGTC |
| | | CGACC | CCATTT |
| human | VE- | CTTCACCCAGACCAA | AATGGTGAAAGCGTC |
| | cadherin | GTACACA | CTGGT |
| human | CLDN5 | AAGTGTACGACTCGG | AAACAGGTAGAGCAC |
| | | TGCTG | GCCTC |
| human | NOS3 | AAAGACAAGGCAGCA | TCCACGATGGTGACT |
| | | GTGGAAAT | TTGGCTA |
| human | CD63 | CAGCTAGAGAGCCCC | TTCATTCCTCCTTCC |
| | | GGA | ACCGC |
| human | PLSCR1 | GGCAGCCAGAGAACT | GGCAAGTTTGTTTCC |
| | | GTTTTAATC | GGGTG |
| human | TBXAS1 | TGCAGAGCACGGTTC | GTGGAGTACCATTTC |
| | | CC | AGGAGGG |
| human | THBS1 | AGTCGTCTCTGCAAC | ACAGGCATCCATCAA |
| | | AACCC | TTGGACA |
| human | ALB | CGCTATTAGTTCGTT | TTTACAACATTTGCT |
| | | ACACCA | GCCCA |
| human | CYP1A2 | CGGACAGCACTTCCC | AGGCAGGTAGCGAAG |
| | | TGAGA | GATGG |
| rat | Gapdh | ACCACAGTCCATGCC | TCCACCACCCTGTTG |
| | | ATCAC | CTGTA |
| rat | Cd63 | CATCAAGAAGCGTCG | ACCTGAACTGCTACG |
| | | GGGA | CCAAT |
| rat | Plscr1 | CTGCGAGGCTTTAGG | CTCTGAGGTCTGCAA |
| | | GAGAG | GGTGG |
| rat | Thbs1 | TAGCTGGAAATGTGG | AGCAAGCATCAGGCA |
| | | TGCGT | CTTCT |
| rat | α-Sma | CATCACCAACTGGGA | TCCGTTAGCAAGGTCG |
| | | CGACA | GATG |
| rat | Vimentin | CACTCACCTGCGAAG | TGGTATTCACGAAGGT |
| | | TGGAT | GGCG |
| rat | Tgf- | CTGCTGACCCCCACT | AGCCCTGTATTCCGTC |
| | beta1 | GATAC | TCCT |
| rat | Timp1 | TGCTCAAAGGATTCG | AGCAGGGCTCAGATTA |
| | | ACGCT | TGCC |

12. Western Blot

Proteins were extracted using PRO-PREP (iNtRon Biotechnology, Korea) and whole cell lysates were sonicated. To extract proteins from tissue samples, the tissue was homogenized with steel beads and PRO-PREP, and then sonicated. After centrifugation, the supernatant was transferred to a new tube and analyzed. Protein concentration was quantified using a DC analysis kit (Bio-Rad, USA). Equal amounts of protein (10 μg) were loaded on an 8 to 15% acrylamide Tris-glycine gel. A target protein was examined with the following primary antibodies; anti-GAPDH (AB2302, Millipore), anti-Integrin beta 3 (#13166, Cell Signaling Technology), anti-Akt (#4691, Cell Signaling Technology), anti-phospho-Akt (#9271, Cell Signaling Technology), anti-cleaved Caspase-3 (#9664, Cell Signaling Technology), anti-Galactosidase alpha (GTX101178, GeneTex), anti-Fibronectin (ab2413, Abcam), anti-Collagen IV (abl9808, Abcam), anti-Laminin (ab11575, Abcam), anti-CK18 (MAB3234, Millipore), anti-CYP2E1 (CSB-PA006425EA01HU, CUSABIO, China) and anti-AFP (A0008, DAKO). After incubation with a primary antibody at 4° C. overnight, the HRP-conjugated secondary antibody was applied. After probing with an Amersham Enhanced chemiluminescence detection kit (GE Healthcare, USA), specific protein bands were derived by FluorChem HD2 (Alpha Innotech., USA).

13. Tissue Examination

Samples were washed three times with PBS and fixed in 4% paraformaldehyde at 4° C. overnight. Tissues were embedded in paraffin and serially cut into a thickness of 10 μm. After deparaffinization of tissue sections, a series of descending concentration of ethanol ranging from 100% to 70% were used for hydration. Subsequently, tissue slices were stained with H&E and a picrosirius red solution (0.1% Direct Red 80 and 0.1% Fast Green FCF). All reagents were purchased from Sigma Aldrich. PAS staining was performed using a PAS staining kit (ab150680, Abcam). After staining, sections were rinsed with tap water, dehydrated using a series of ascending concentrations of ethanol, and mounted. Visualization was performed using Nikon NIS-Elements software and an optical microscope (Nikon), and quantification of fibrosis was performed using Image J software.

14. Resazurin Reduction Perfusion Assay

PrestoBlue Cell Viability Reagent (A13261, Invitrogen), which is a non-toxic resazurin-based solution, was used according to the manufacturer's instructions. To plot a standard curve for HUVECs, 0.02, 0.05, 0.1, 0.2, 0.5, 1 and 2 million cells were seeded in 6 well plates. After culture for 6 hours, cells were washed and medium was replaced with 2 ml of PrestoBlue reagent mixed with complete EGM-2 (ratio of 1:20). After incubation at 37° C. for 1 hour, the medium was collected, and then the absorbance at a wavelength of 570 nm was measured using a microplate reader. According to the standard curve, 30 ml of a PrestoBlue-intermediate mixture was perfused into the engineered construct at 37° C. for 1 hour. Harvested media were analyzed as described above.

15. FITC-Conjugated Dextran Perfusion Assay

To evaluate the permeability of re-endothelialized vessels in engineered tissues, perfusion of 500 kDa FITC-dextran (FD500S, Sigma Aldrich) through the portal vein was used. 25 mL of FITC-dextran (0.2 mg/mL) was administered at 20 mmHg. Dextran in a fluid discharged from the inferior vena cava was considered intravascular dextran, whereas dextran in the peripheral blood was considered extravascular dextran. The amount of total dextran was determined by fluorescence intensity (absorbance at 490 nm) multiplied by the amount of fluid discharged.

16. TUNEL Assay

To detect apoptotic cells from engineered constructs, the ApopTag Red In situ Apoptosis Detection Kit (Millipore) was used. Tissue sections were deparaffinized and digested with proteinase K. After an equilibration buffer was applied to the sections, the sections were probed with TdT enzyme in an incubator at 37° C. for 1 hour. After washing with stop/wash buffer, anti-digoxigenin (rhodamine-conjugate) was applied to the sections. Subsequently, these sections were washed with PBS, and then the nuclei were stained with DAPI. TUNEL-positive cells were detected under a confocal microscope.

17. Quantitation of Secreted NO and VEGF

The concentration of NO secreted from the bioengineered constructs was measured using the NO Plus detection kit (iNtRON). Conditioned media were harvested at the indicated time points and cell debris was removed through centrifugation. Thereafter, NO was measured from the supernatant of the conditioned media according to the manufacturer's instructions. Absorbance at a wavelength of 540 nm was measured using a microplate reader. Constructs were cultured in endothelial basal medium-2 (EBM-2) without growth factors and exposed to the human VEGF Quantikine ELISA kit (DVE00, R & D Systems, USA). Twelve hours later, the supernatant was obtained for VEGF quantification of the conditioned media from each engineered construct. Absorbance at a wavelength of 450 nm was measured using a microplate reader.

18. Ex Vivo Human Blood Perfusion Assay

Scaffolds repopulated with 1×10⁷ HUVECs were maintained in EGM-2 supplemented with 10% FBS and antibiotics for 7 days before perfusion with human blood. Heparinized human blood obtained from the Korean Red Cross Central Blood Center was mixed with culture medium (1:1) and perfused through the portal vein of each scaffold. At the indicated time points, blood tube effluent was collected and the number of platelets in the blood perfusate was counted using an Advia 2120i hematology system (Siemens Healthineers, Germany). 24 hours after perfusion, samples were washed with PBS and subjected to gene expression analysis and immunostaining to evaluate the degree of thrombus development within the scaffolds. cDNA was synthesized from the extracted RNA and then amplified by PCR using primers targeting the thrombosis gene. Further, paraffin-embedded tissue blocks were sectioned and stained with integrin αIIb in accordance with the procedure described in the immunofluorescent staining method.

19. Albumin/Urea ELISA Analysis

The amount of albumin protein secreted from the constructs in the conditioned media was determined using a human albumin ELISA kit (ab108788, Abcam). Conditioned media from VBHL constructs were harvested at the indicated time points. After centrifugation, the concentration of secreted albumin in the supernatant was quantified using ELISA according to the manufacturer's recommendations. Absorbance at a wavelength of 450 nm was measured using a microplate reader. In addition, the amount of urea secreted from the supernatant collected in the conditioned media was measured. The concentration of urea was quantified by measuring the absorbance at a wavelength of 520 nm using the QuantiChrom Urea Assay Kit (BioAssay Systems, USA).

20. In Vivo Reperfusion of Vascularized Liver Constructs

First, decellularized liver constructs were prepared by catheterization as follows: 22G catheter-portal vein, 26G catheter-bile duct and 20G catheter-inferior vena cava. VBHL constructs were produced as described in Experimental Methods Part 6. The VBHL constructs were maintained in a bioreactor for 21 days and then directly connected to a host renal circulation system by catheters with various sizes. For the above procedure, the left kidney was exposed by anesthetizing a 1-year-old healthy rat. After fixation of the renal artery and vein, each 24G catheter was catheterized. Catheters disposed in the portal vein and inferior vena cava of the construct were connected to catheters disposed in the renal artery and vein, respectively. Each connection part was fixed with Vetbond glue (3M, USA). After a vascular clamp was removed, the blood was perfused in vivo for 2 hours. Thereafter, an additional analysis of the harvested liver constructs was performed.

21. TAA-Induced Chronic Liver Injury Rat Model and Transplantation of VBHL Constructs

To evaluate the in vivo function of VBHL constructs, chronic liver injury was induced in 4-week-old female rats. After 0.3 g/L TAA (Sigma Aldrich) containing drinking water was continuously administered to rats for 12 weeks, induced liver fibrosis was analyzed. ALT and AST levels in rat serum were measured using the ALT Activity Colorimetric assay kit (K752-100, Biovision, China) and AST Activity Colorimetric assay kit (K753-100, Biovision). After liver cirrhosis was induced in rats for 8 weeks, the liver was exposed by performing a rat laparotomy. Subsequently, fibrous capsules were removed from the VBHL constructs of each group and then transplanted and fixed between the medial and right lateral lobes of the host liver. VBHL constructs were produced as described in Experimental Methods Part 6, using decellularized rat livers. After 4 weeks, the livers of hosts transplanted with liver constructs were harvested again and subjected to additional analyses. Meanwhile, serum samples were obtained before and after surgery.

### Example 1. Characterization of Anti-CD31 Aptamer

As illustrated in FIG. TA, an anti-CD31 aptamer capable of specifically binding to the CD31 protein was produced, and then the binding strength between CD31-expressing cells and the aptamer was verified using a flow cytometer.

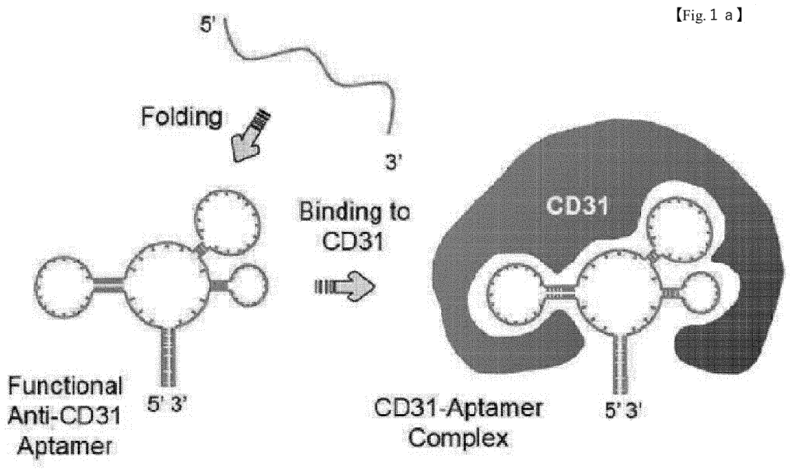

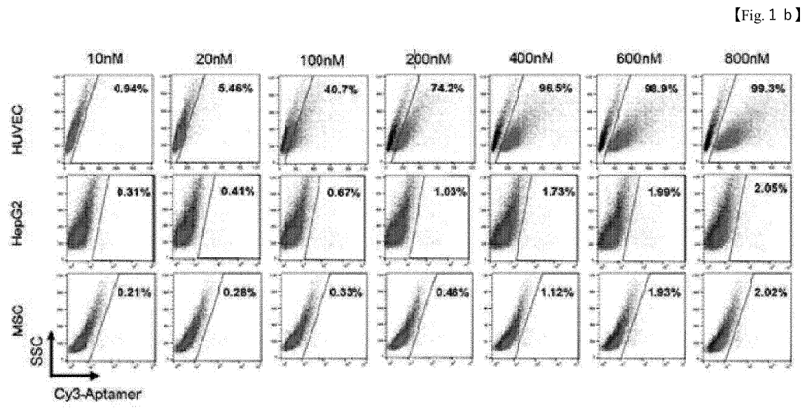

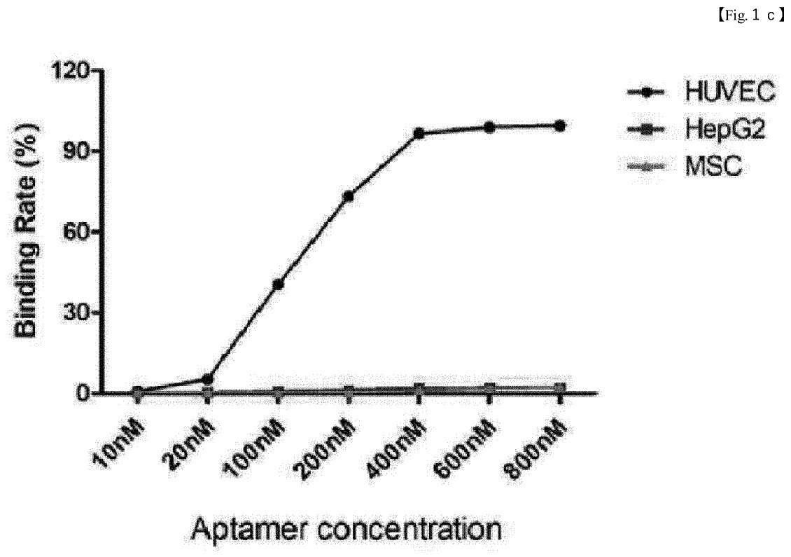

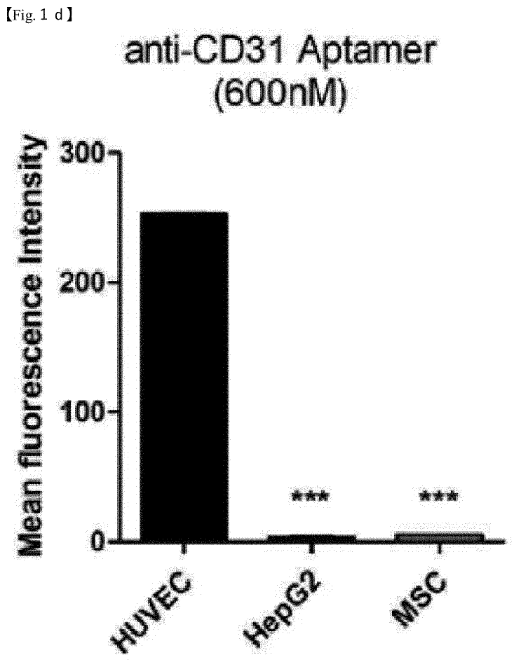

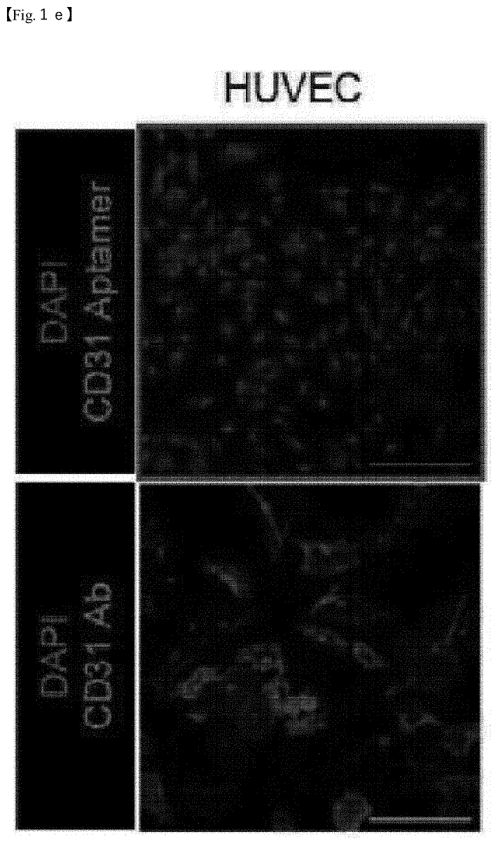

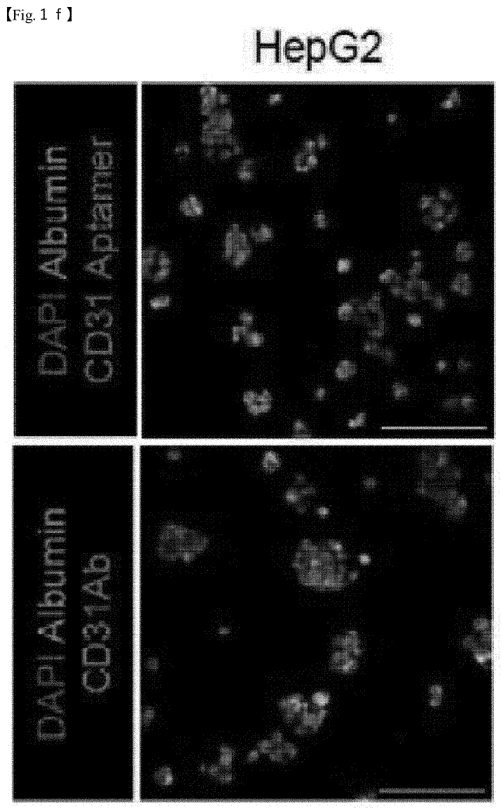

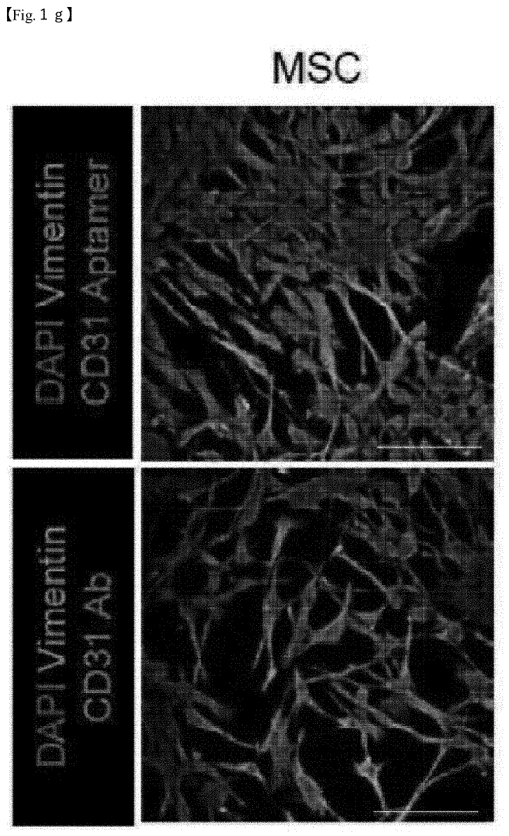

As a result, as illustrated in FIGS. 1B and 1C, 600 nM and 800 nM aptamers were able to bind to about 99% of vascular endothelial cells (HUVECs), and HepG2 and MSCs that do not express CD31 were bound to about 2% of the cells. In addition, when the binding strength with vascular endothelial cells according to the aptamer concentration was quantified, the binding strength was saturated at a 600 nM aptamer, so that the aptamer concentration condition was optimized at 600 nM.

Furthermore, as illustrated in FIGS. 1E to 1G, it was confirmed through a cell immunostaining method that the 600 nM aptamer binds to HUVECs, but not to HepG2 and MSCs. Based on the above results, it was verified that the anti-CD31 aptamer specifically binds to CD31.

### Example 2. Evaluation of Adhesion Ability of Vascular Endothelial Cells During Aptamer Treatment and Confirmation of Effects on Vascular Endothelial Cells

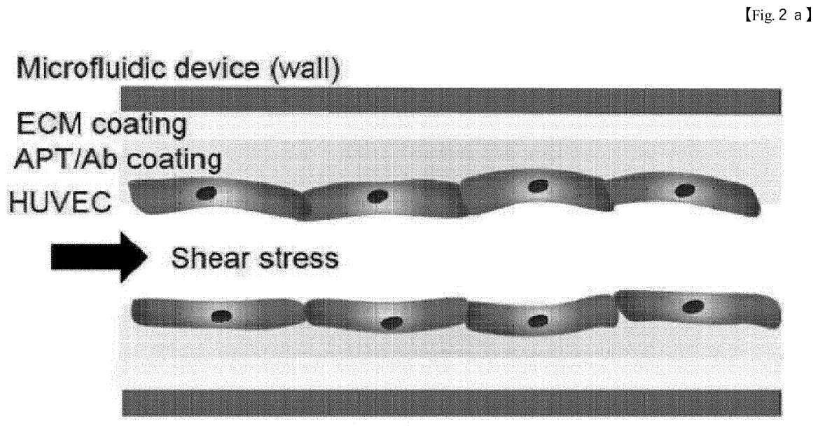

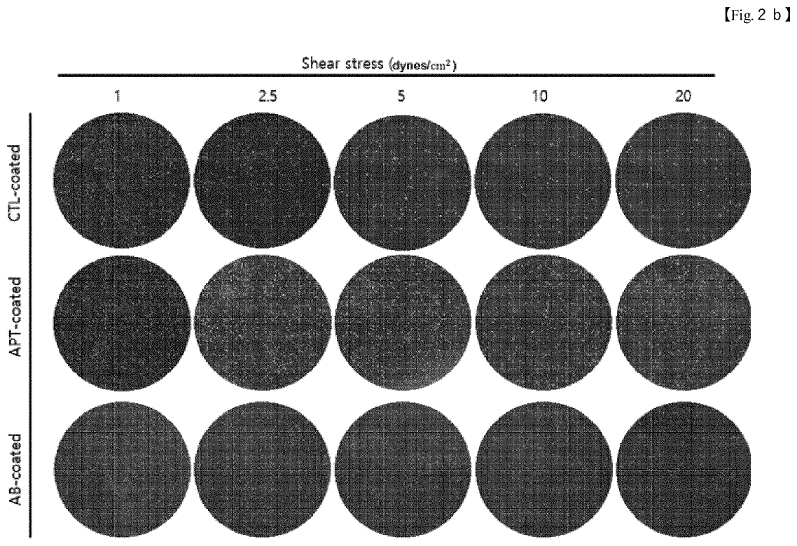

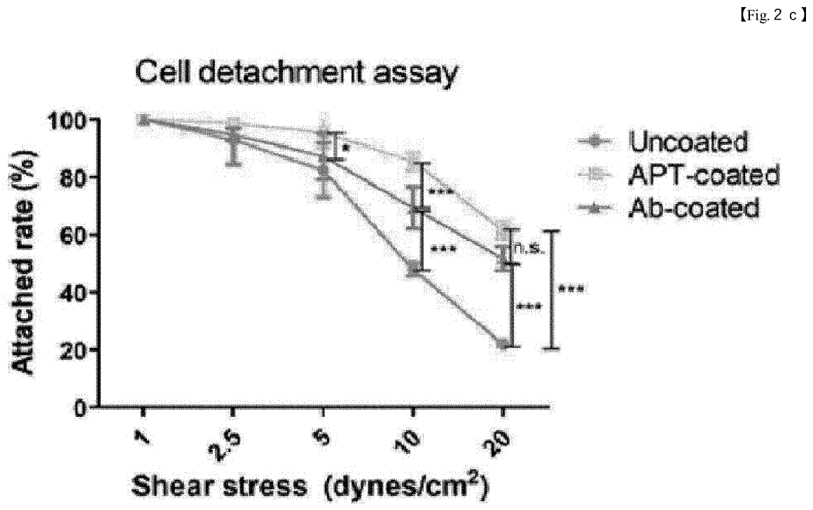

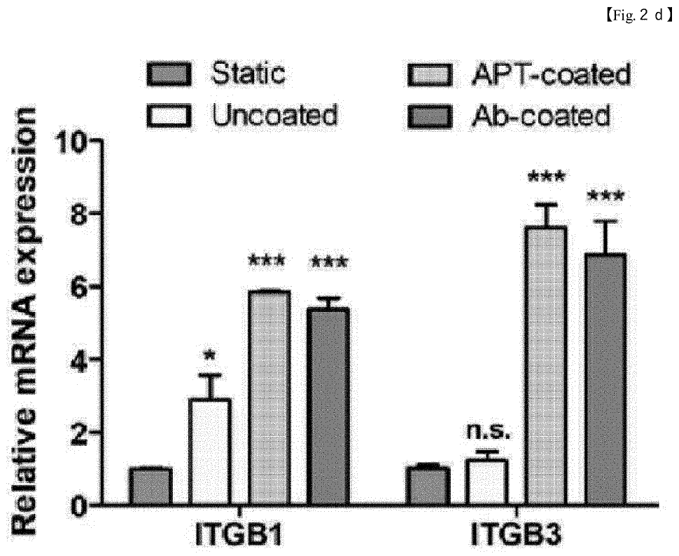

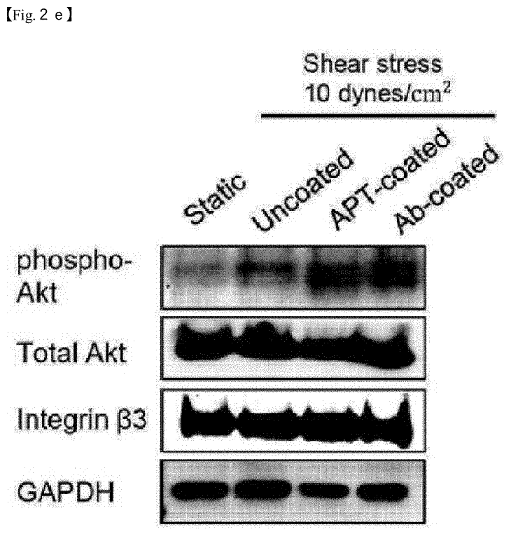

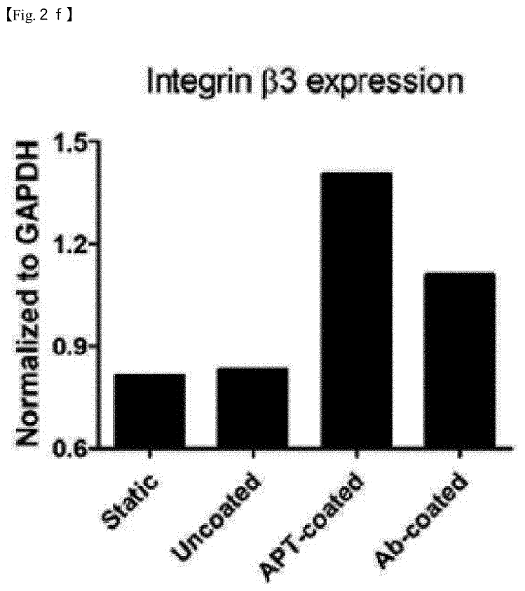

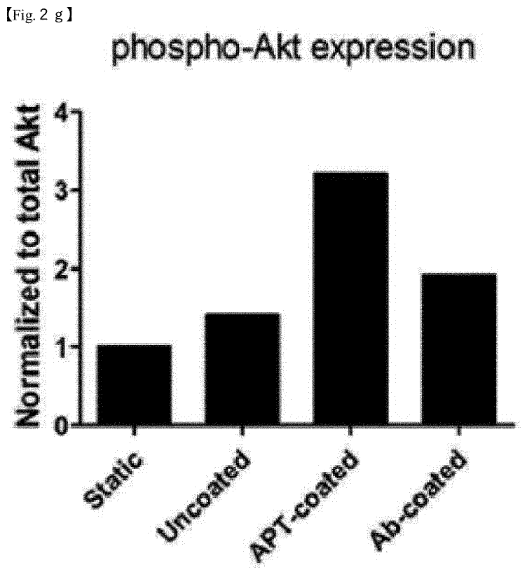

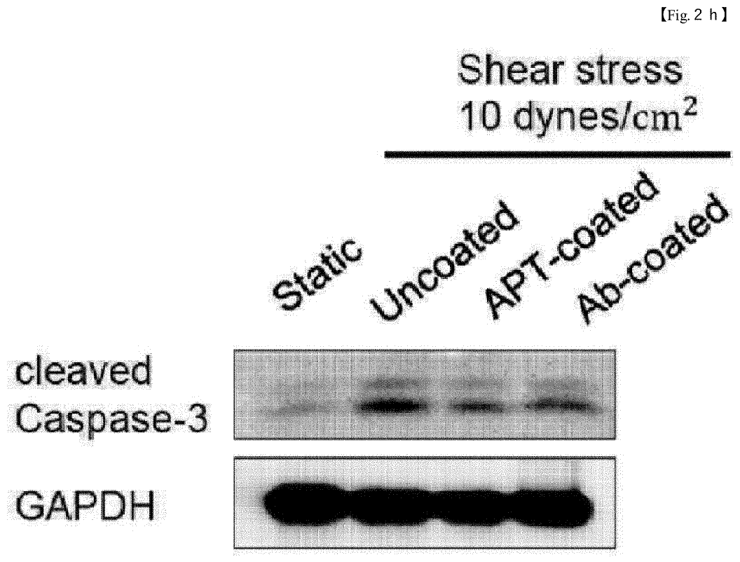

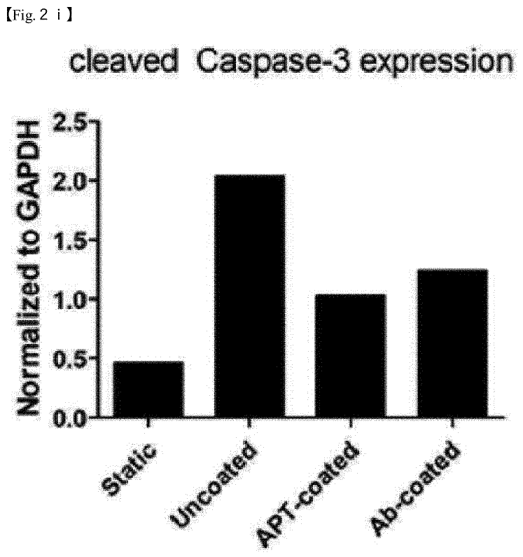

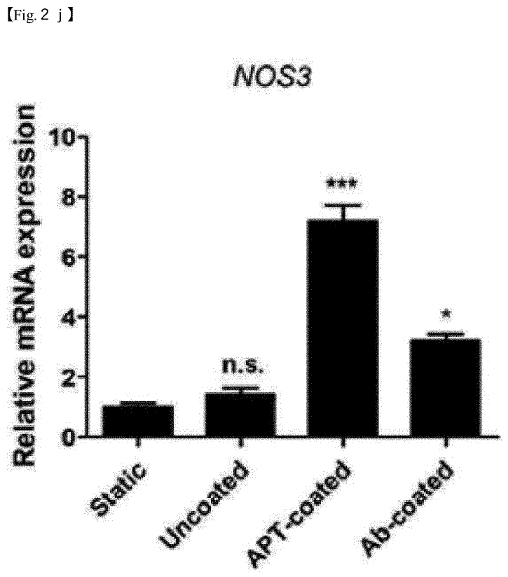

As illustrated in FIG. 2A, the inside of the microfluidic device was coated with extracellular matrix (ECM) components, and then coated with an anti-CD31 aptamer (APT-coated) and an anti-CD31 antibody (Ab-coated, positive control), respectively, and HUVECs were injected, and then cell adhesion ability was evaluated while flowing the liquid at a constant speed.

As a result, as illustrated in FIGS. 2B and 2C, more than 80% of the cells in the APT-coated group adhered strongly when a shear stress of 10 dynes/cm² was applied compared to the uncoated group, and showed a high adhesion ability of 61.5% even when a strong shear stress of 20 dynes/cm² was applied. The Ab-coated group, which is a positive control, showed an adhesion ability of 51% when treated with a shear stress of 20 dynes/cm². Through this, it is shown that the anti-CD31 aptamer enhances the adhesion ability of vascular endothelial cells with higher efficiency than the anti-CD31 antibody.

Furthermore, to investigate a mechanism that mediates the difference in adhesion ability of HUVECs by each coating agent, the mRNA expression pattern was analyzed by extracting RNA from cells in the microfluidic device.

It is known that integrin proteins consist of an extracellular domain, a transmembrane domain, and an intracellular domain, and when the extracellular domain binds to the extracellular matrix, signals are transmitted into the cell to activate various signaling systems. Thus, it was confirmed whether or not the integrin protein can be activated by shear stress.

As a result, as illustrated in FIGS. 2E to 2G, it was confirmed that not only integrins but also Akt signaling, which is a lower signaling system, were activated in HUVECs subjected to a shear stress of 10 dynes/cm² in a microfluidic device actually coated with an aptamer. Akt signaling has been reported to be associated with cell survival and angiogenesis potential in vascular endothelial cells. Accordingly, as illustrated in FIG. 21, it was also confirmed that the expression of cleaved caspase-3, which is a cell apoptosis marker, was reduced in the APT-coated group.

### Example 3. Verification of Efficacy of Aptamers During Reconstruction of Vascular Structures by Re-Cellularization of Vascular Endothelial Cells in Decellularized Scaffolds

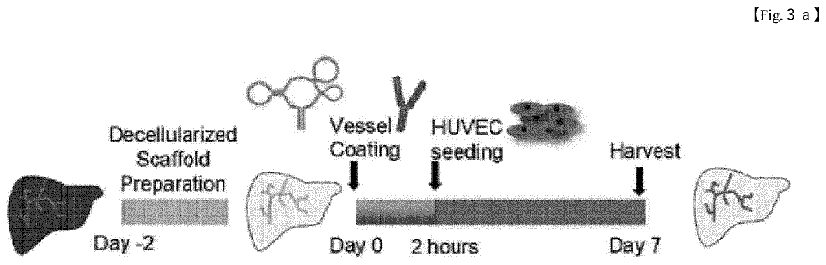

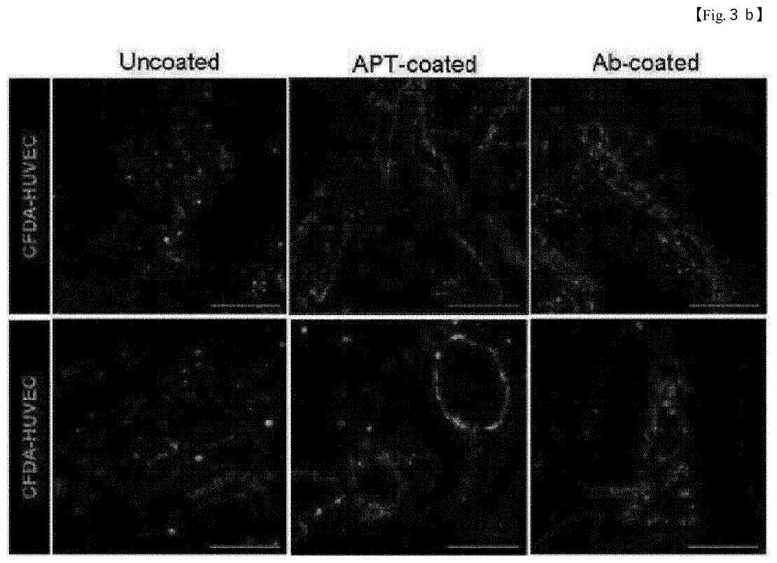

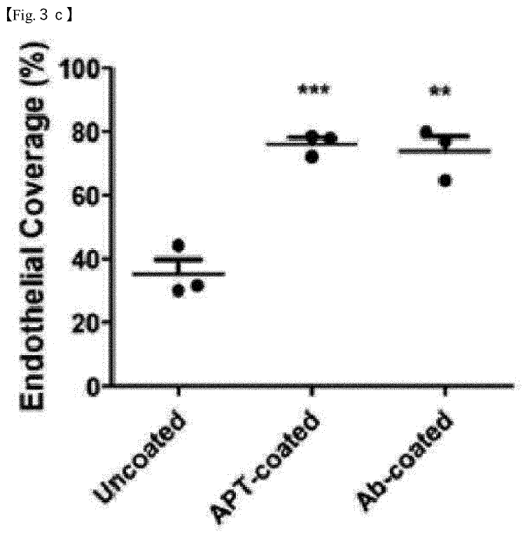

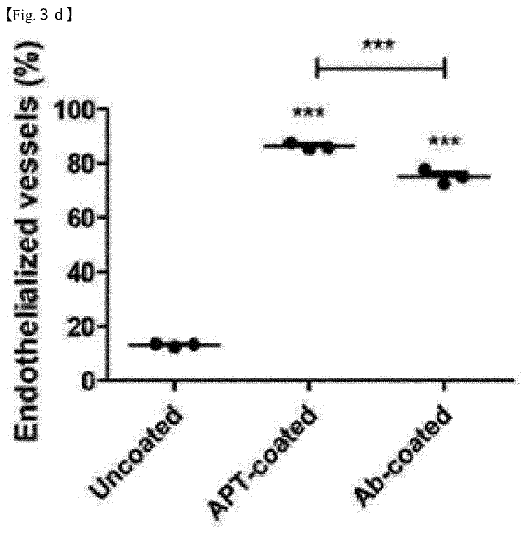

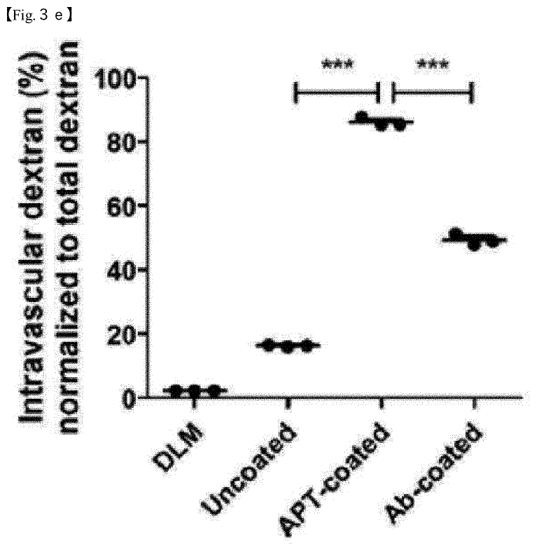

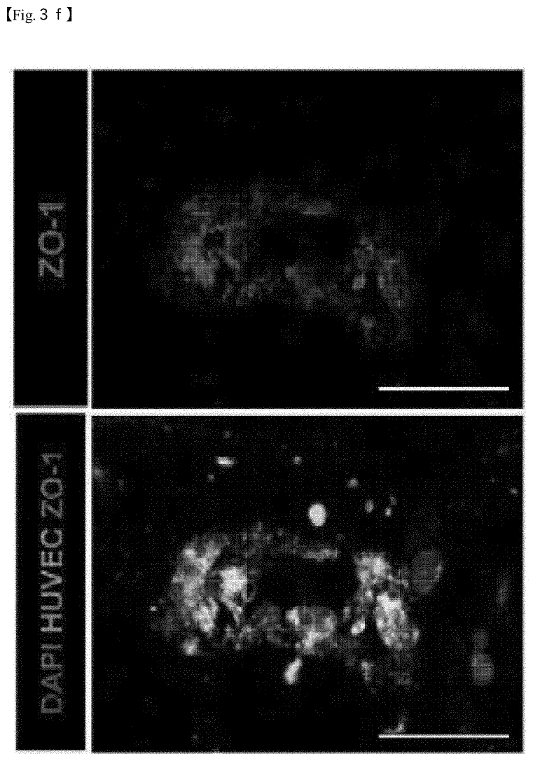

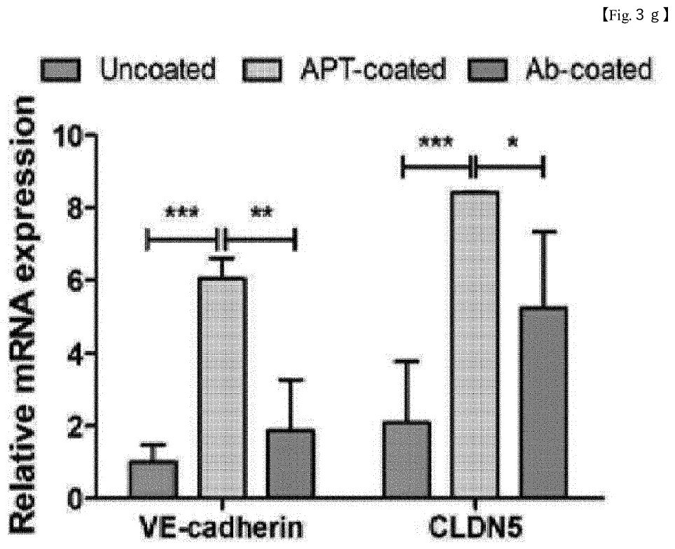

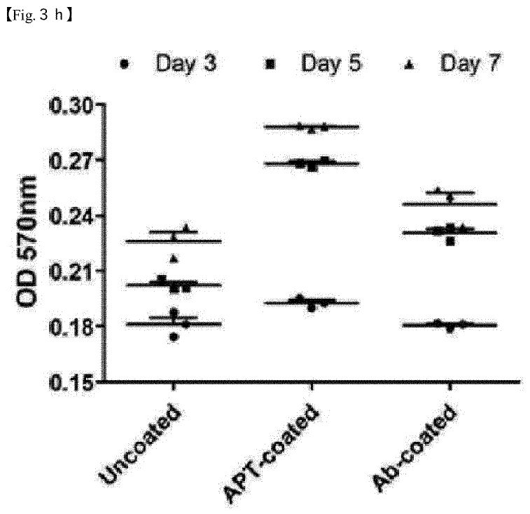

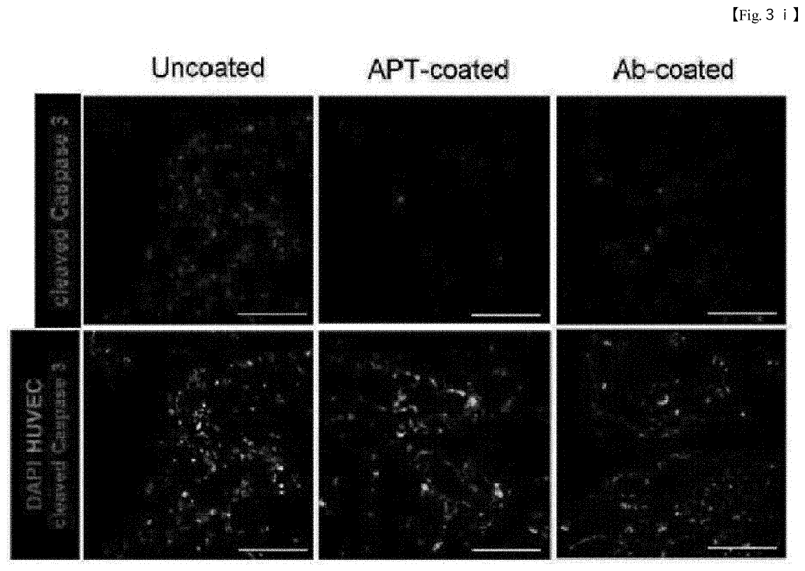

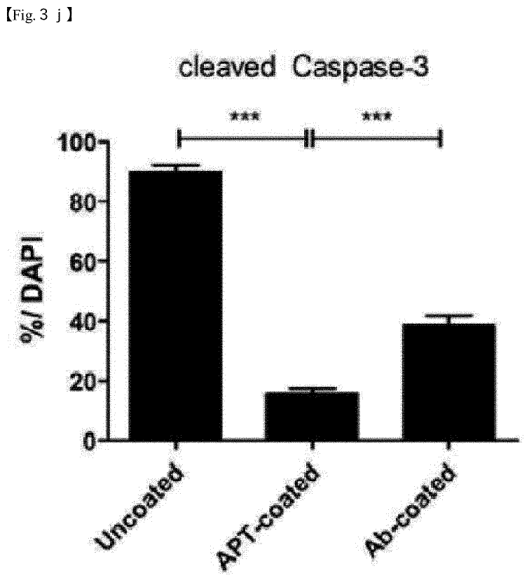

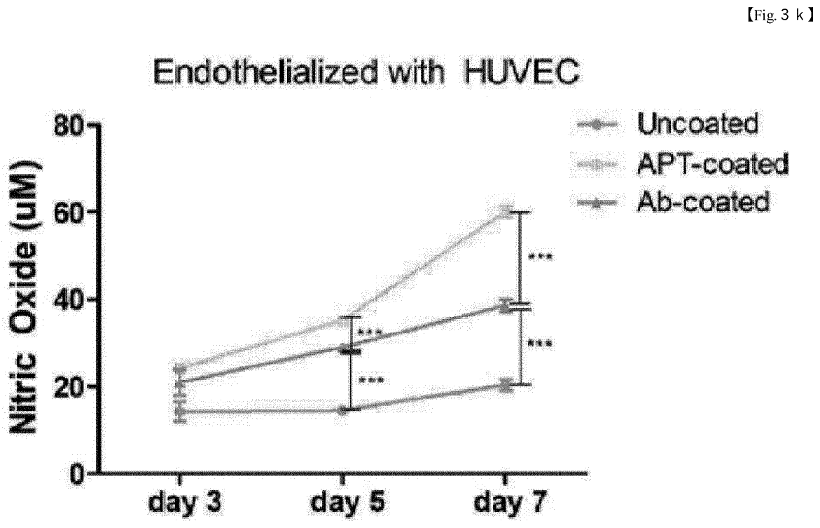

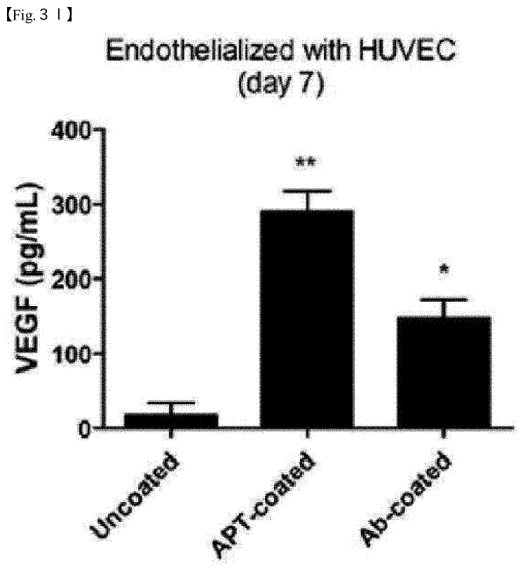

As illustrated in FIG. 3A, a decellularized scaffold was produced from the rat liver, and then the inner wall of the blood vessel was coated with a coating agent. HUVECs labeled with CFDA (green) were recellularized, and then cultured for 7 days and each reconstructed vessel structure was confirmed.

As a result, as illustrated in FIG. 3B, it was confirmed that vascular endothelialization was well performed within the scaffold treated with the anti-CD31 aptamer as a coating agent.

Furthermore, as illustrated in FIGS. 3C and 3D, it was confirmed that about 80% endothelialization per blood vessel was achieved in the APT-coated group, and about 80% vascular endothelialization was achieved within the scaffold. Through this, it was confirmed that endothelialization efficiency was maximized in the aptamer-treated group compared to the uncoated group in which coating was not performed.

Further, as illustrated in FIG. 3E, it was confirmed that when dextran was injected through a portal vein catheter to evaluate the barrier function of reconstructed blood vessels, the amount of intravascular dextran that did not leak out of the blood vessel was significantly increased in the APT-coated group. Therefore, it was verified that when the scaffold was treated with the anti-CD31 aptamer as a coating agent, a highly functional vascular structure in which the barrier function was well maintained was efficiently reconstructed in the decellularized scaffold.

In addition, since the viability of cells cultured during in vitro culture can significantly affect the functionality of reconstructed artificial organs, the degree of apoptosis of cultured cells was analyzed through an immunostaining method.

As a result, as illustrated in FIGS. 31 and 3J, cell apoptosis was reduced in the APT-coated group compared to the uncoated group or the anti-CD31 antibody-coated group.

Thereafter, enzyme-linked immunosorbent assay (ELISA) was performed to verify the blood vessel-specific functionality of a reconstructed vascularized organ.

As a result, as illustrated in FIGS. 3K and 3L, it was confirmed that when the amounts of nitric oxide (NO) and VEGF secreted from the HUVEC-recellularized scaffolds were analyzed, the amounts of NO and VEGF secreted in the APT-coated group was significantly increased compared to the other groups.

Overall, it was verified that when the anti-CD31 aptamer was used as a coating agent, not only revascularization efficiency and barrier function, but also vascular viability and angiogenesis potential were enhanced.

### Example 4. Evaluation of Thrombus Formation in Scaffold by Ex Vivo Human Blood Perfusion

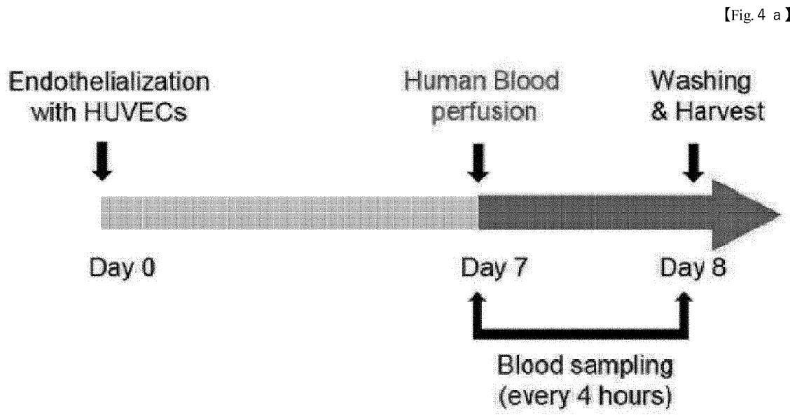

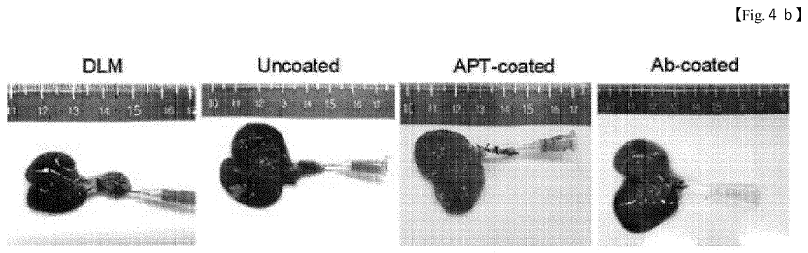

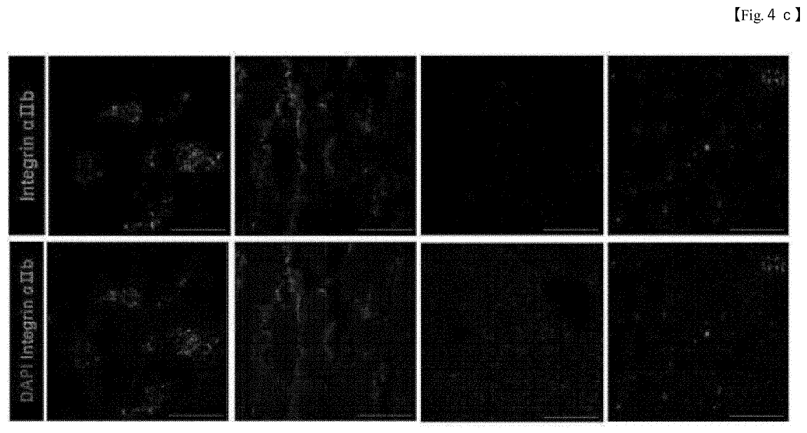

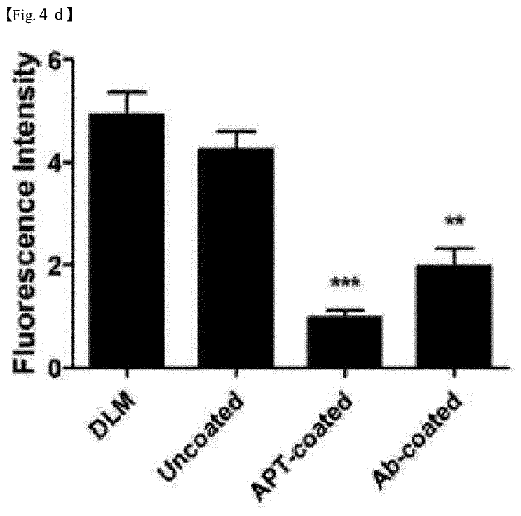

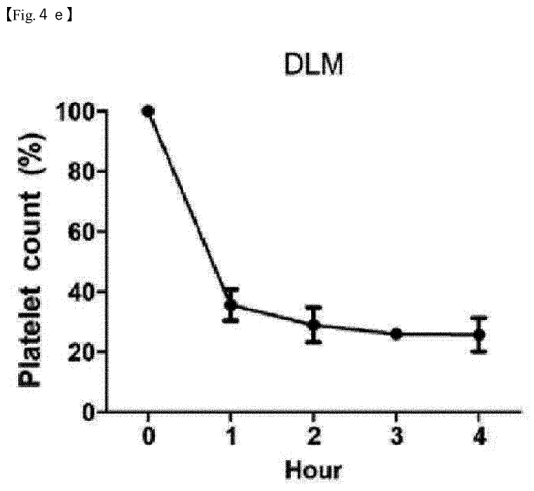

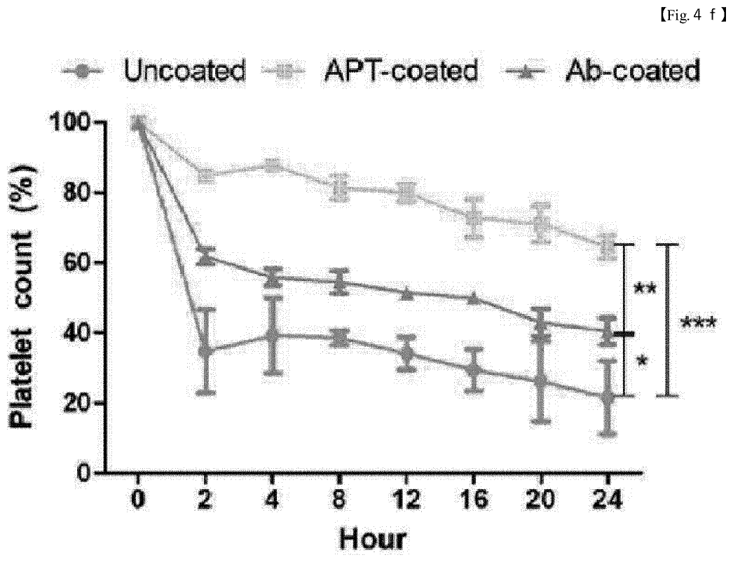

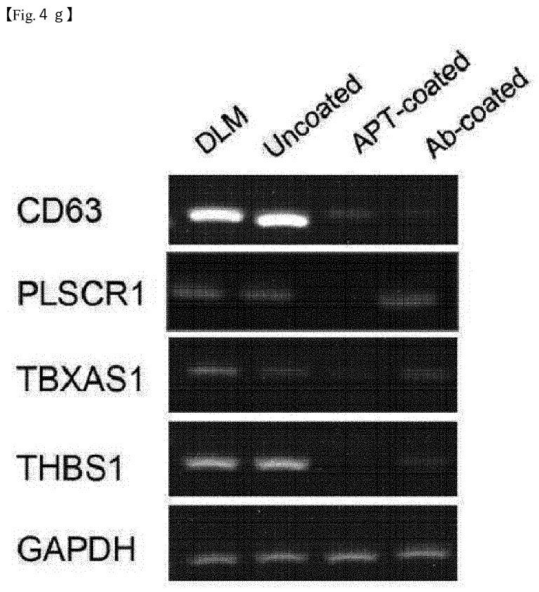

As illustrated in FIG. 4A, in order to closely verify the degree of vascular reconstruction, the degree of thrombus formation was evaluated ex vivo by perfusing human blood into a scaffold in which HUVECs were recellularized and cultured for 7 days. By the analysis method, the degree of thrombus formation when artificial organs were implanted in actual humans could be evaluated.

As a result, as illustrated in FIG. 4B, thrombus formation was visually reduced in the APT-coated group. In addition, as illustrated in FIGS. 4C and 4D, it was confirmed through immunostaining that the expression of integrin αIIb, which is a thrombus marker, was decreased in the APT-coated group.

Further, as illustrated in FIGS. 4E and 4F, it was confirmed that when the number of platelets was quantified in the perfusate after blood perfusion, the number of platelets remaining in the perfusate of the decellularized liver matrix (DLM) group, which was not re-endothelialized, was decreased to less than 20% after 4 hours due to platelet aggregation, whereas about 60% of platelets remained in the APT-coated group even 24 hours after perfusion.

In addition, as illustrated in FIG. 4G, the expression of markers (CD63, PLSCR1, TBXAS1 and THBS1) associated with platelet aggregation was decreased in the APT-coated group.

Based on the above results, it was verified that aptamer coating enabled the formation of blood vessels capable of perfusion through more efficient vascular endothelialization, and minimized thrombus formation after blood perfusion.

### Example 5. Production of Vascularized Artificial Liver Using Aptamer and Functionality Evaluation

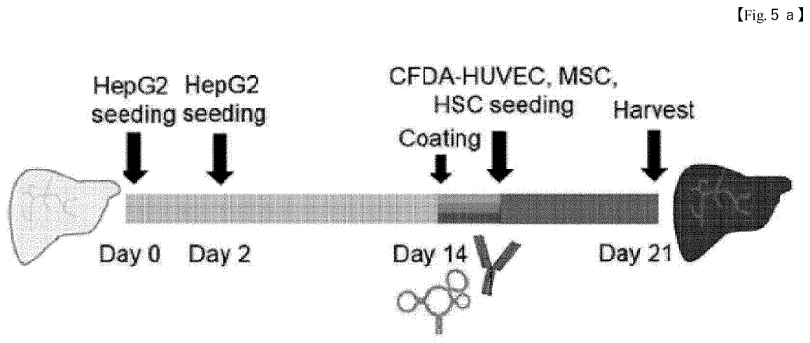

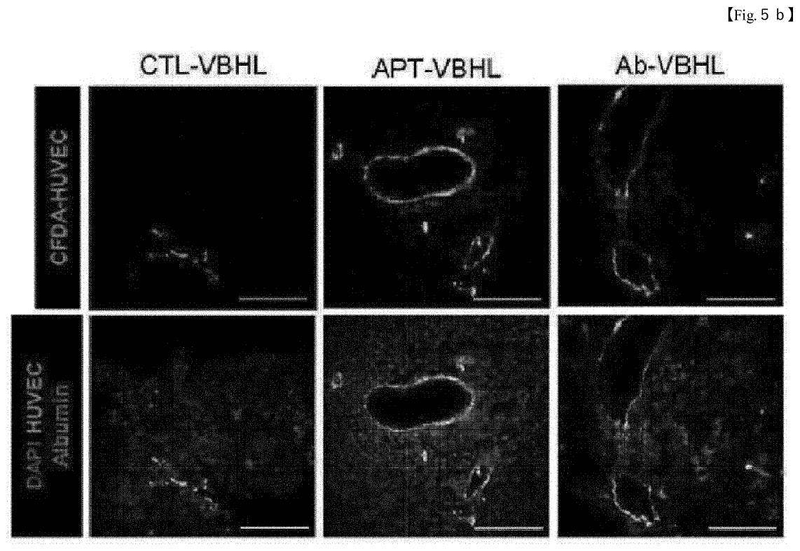

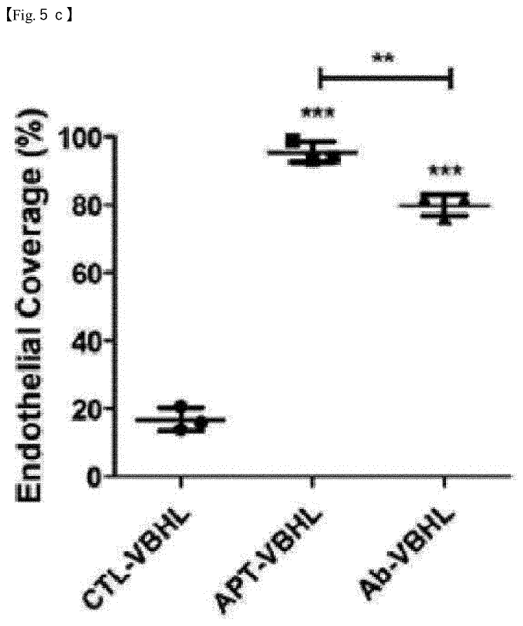

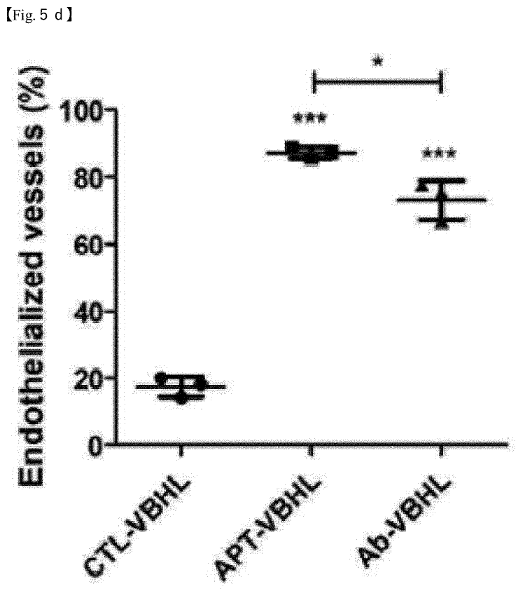

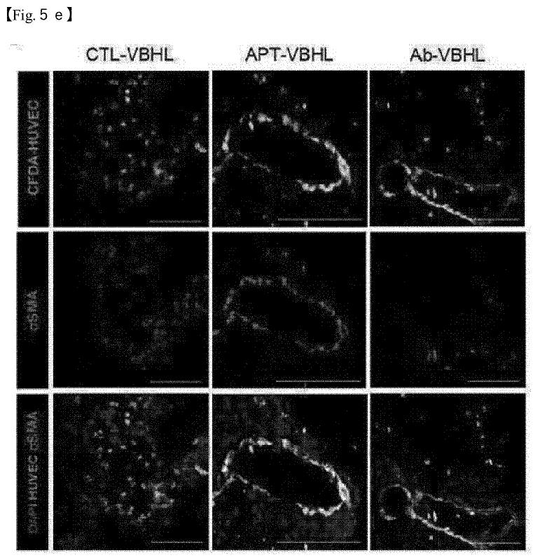

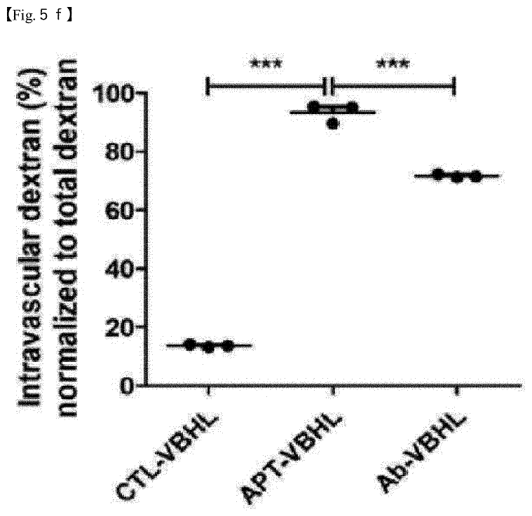

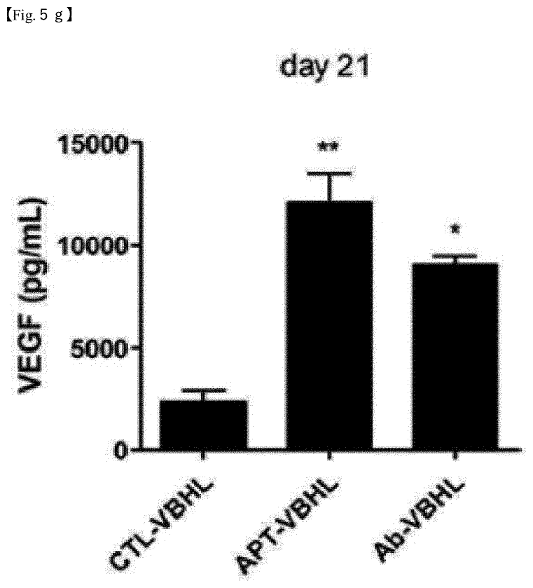

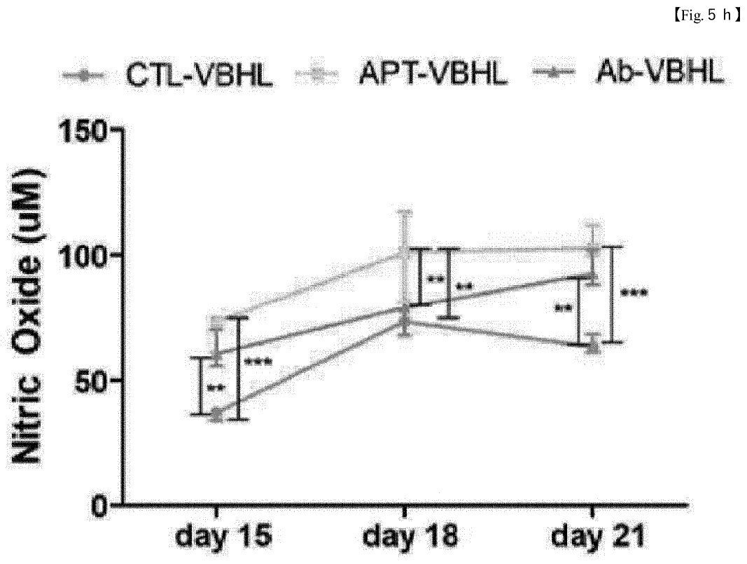

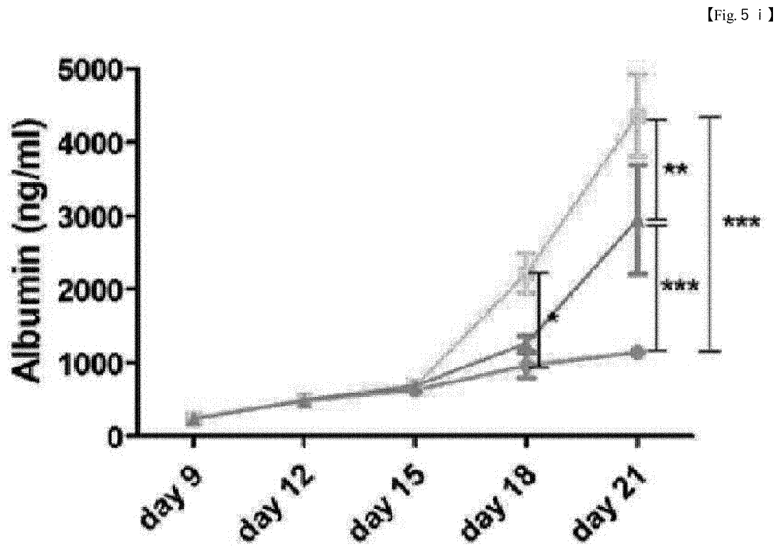

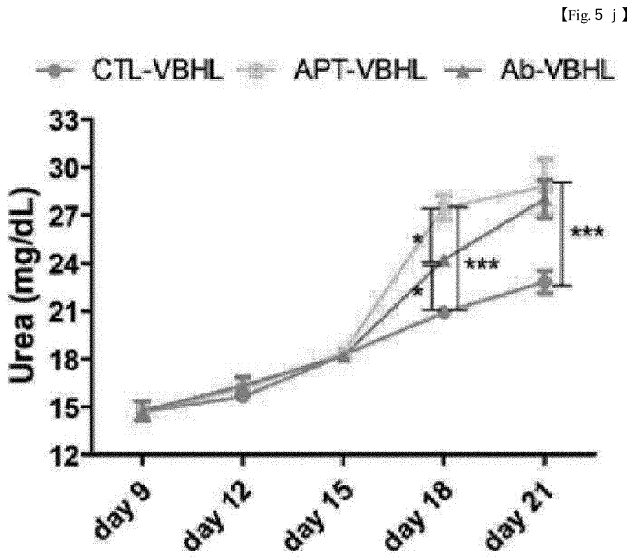

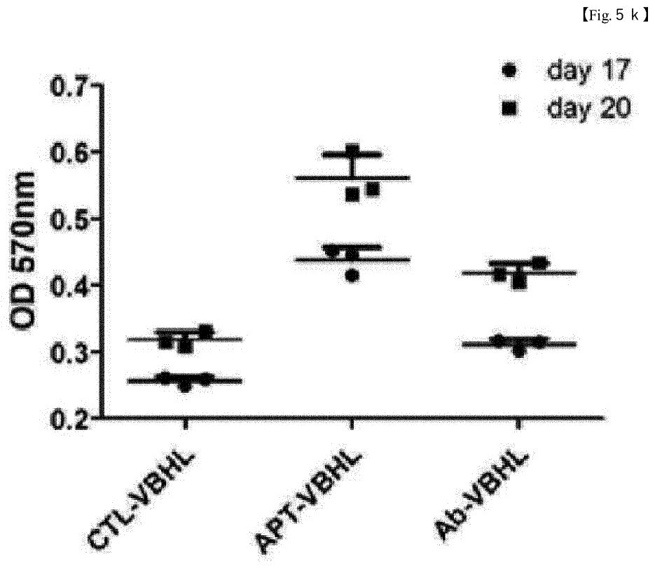

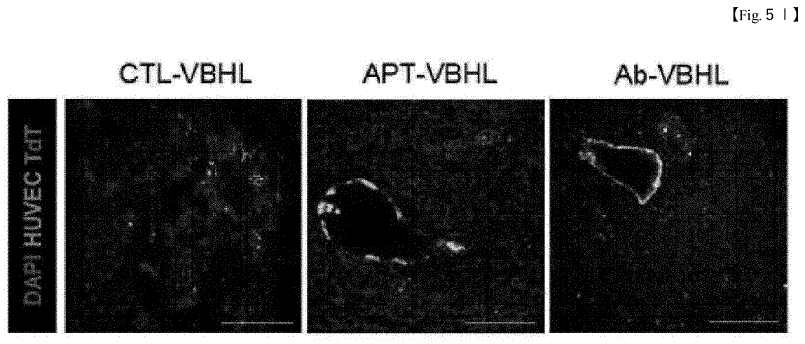

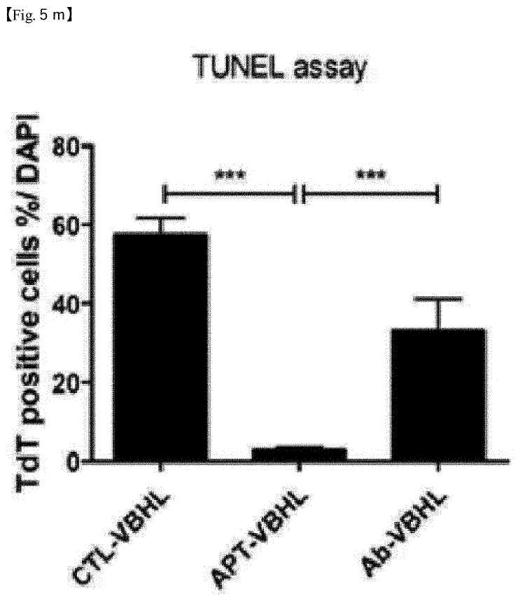

In order to reconstruct a vascularized bioengineered human liver (VBHL) using aptamers, hepatic parenchymal cells (HepG2) and interstitial cells (hepatic stellate cells, mesenchymal stem cells), vascular endothelial cells (HUVEC), and the like were cultured by the method illustrated in FIG. 5A.

As a result, as illustrated in FIGS. 5B to 5D, it was confirmed by an albumin immunostaining method that reconstruction of the parenchymal part of the liver as well as more than 90% of vascular endothelialization was achieved in an artificial liver tissue (APT-VBHL) in which vascular endothelialization was performed after coating with an aptamer.

Furthermore, as illustrated in FIG. 5E, it was confirmed through an aSMA immunostaining method that mesenchymal stem cells were well engrafted in the perivascular region during aptamer treatment. In contrast, it was observed that the perivascular region was not developed in the uncoated or antibody-treated groups.

Further, as illustrated in FIG. 5F, it was confirmed that when dextran was flowed through the hepatic portal vein and intravascular dextran was quantified, dextran was significantly increased in the APT-VBHL group. Through this, it was confirmed that the vascular barrier function of liver tissue was maintained.

Therefore, not only vascular endothelialization but also reconstruction of the perivascular region that constitutes the vascular wall during aptamer treatment could result in obtaining highly functional blood vessels which maintain their barrier function well.

In addition, the degree of cell apoptosis in the artificial liver tissue was quantified through TUNEL assay.

As a result, as illustrated in FIG. 5L, the APT-VBHL group exhibited the least cell apoptosis, which is thus linked to the functionality of the artificial liver.

In addition, the NO production amount and the amount of VEGF secreted from the artificial liver were measured in order to confirm blood vessel-specific functionality.

As a result, as illustrated in FIGS. 5G and 5H, the APT-VBHL group showed excellent functionality compared to the other groups.

Furthermore, ELISA, which quantifies albumin and urea components secreted from the artificial liver, was performed in order to confirm liver-specific functionality.

As a result, as shown in FIGS. 51 and 5J, it was confirmed that the amounts of albumin and urea secreted on day 21 of culture were also the highest in the APT-VBHL group. In particular, since liver functionality showed a difference after blood vessels were reconstructed, it can be seen that highly functional revascularization through aptamer coating plays an important role in maintaining the overall functionality of the artificial liver tissue.

### Example 6. Evaluation of In Vivo Thrombus Formation of Aptamer-Treated Vascularized Artificial Liver

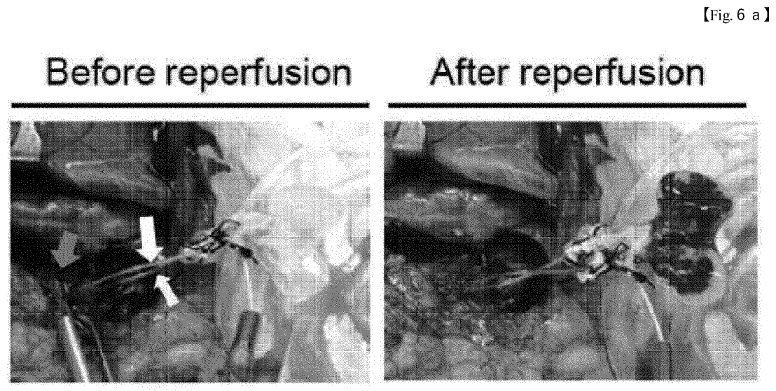

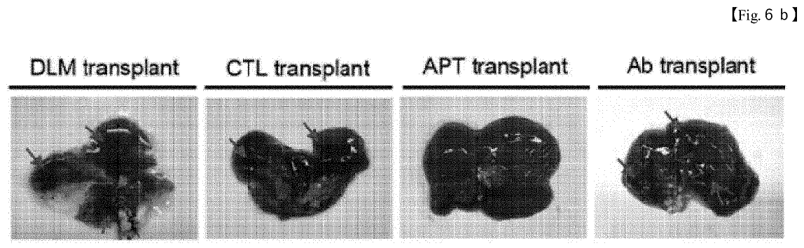

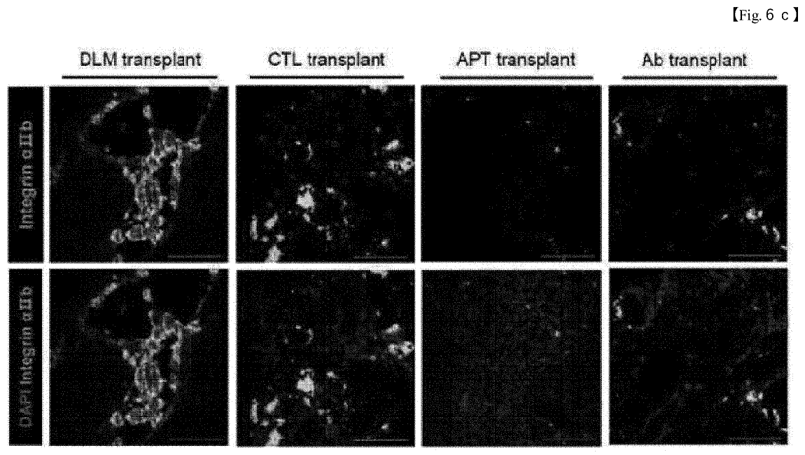

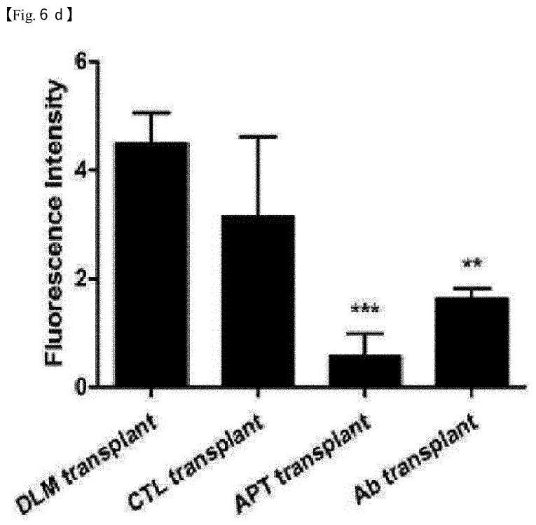

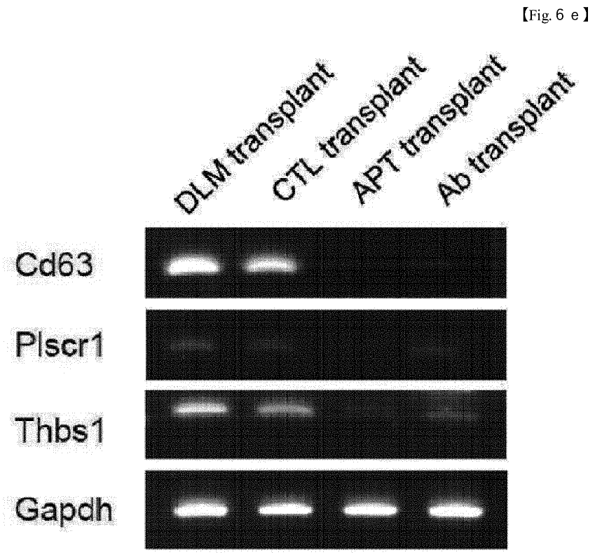

As illustrated in FIG. 6A, the degree of thrombus formation in the vascularized artificial liver in vivo was evaluated by connecting the hepatic portal vein and inferior vena cava of the artificial liver to the renal artery and renal vein of rats, respectively, to perfuse blood in vivo. Specifically, the renal vein (yellow arrow) was connected to the inferior vena cava of the VBHL construct and the renal artery (white arrow) was connected to the portal vein of the construct. After the vascular clamp (blue arrow) was removed, the VBHL construct was reperfused with a renal circulation system in vivo.

As a result, as illustrated in FIG. 6B, thrombus formation was not visually observed in the aptamer-treated artificial liver.

Further, as illustrated in FIGS. 6C and 6D, it was confirmed that the expression of integrin αIIb, which is a thrombus marker, was also reduced.

In addition, as illustrated in FIG. 6E, when the expression of blood coagulation-related mRNA markers (Cd63, Plscr1, and Thbs1) was analyzed, the expression of blood coagulation-related markers was most decreased in the APT-VBHL group.

Therefore, based on the above results, it was verified that an artificial liver culture capable of minimizing thrombus formation in vivo was successfully achieved through aptamer treatment.

Therefore, it is expected that when the aptamer coating agent of the present invention is used, the success rate of artificial liver transplantation can be increased by minimizing the most serious side effects that may occur after artificial liver transplantation.

### Example 7. Evaluation of In Vivo Liver Functionality of Aptamer-Treated Vascularized Artificial Liver

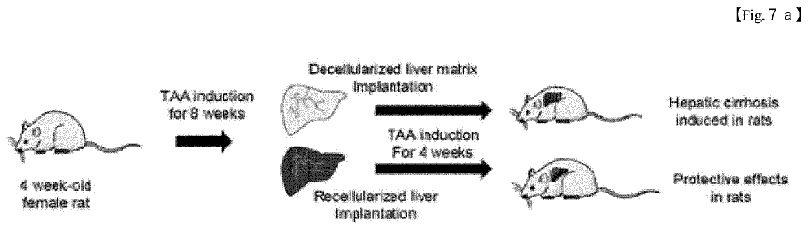

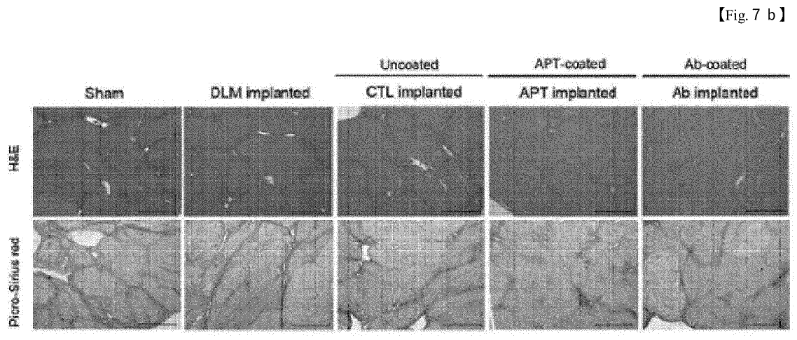

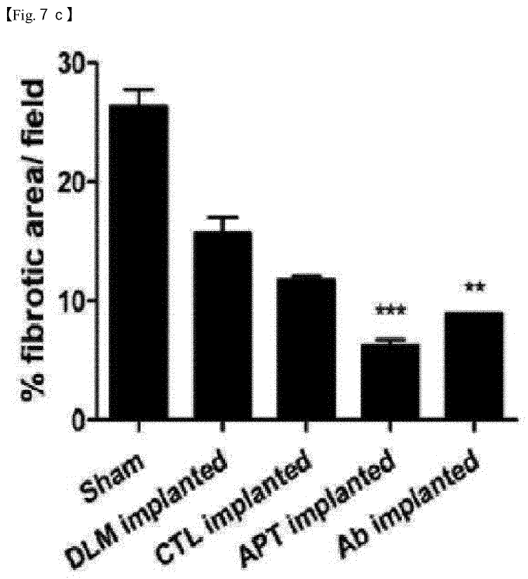

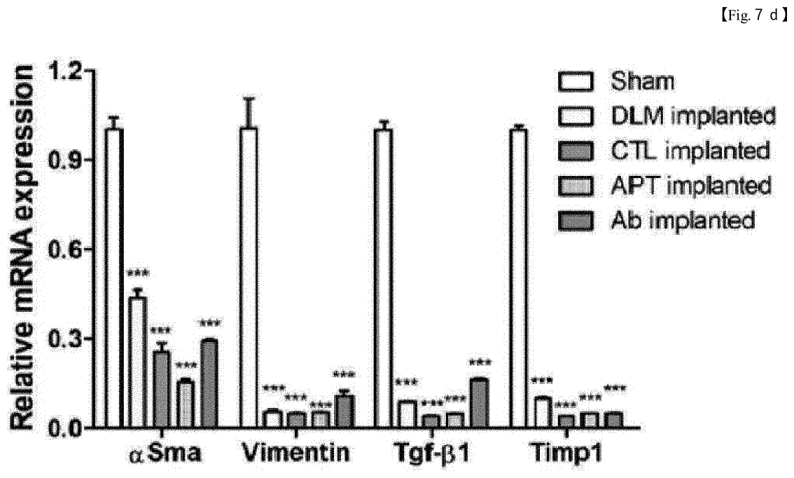

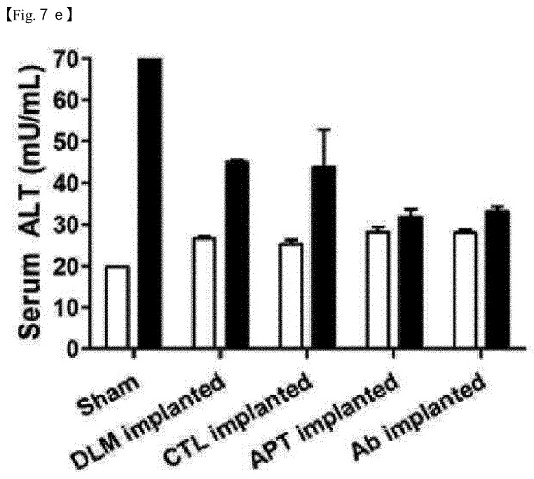

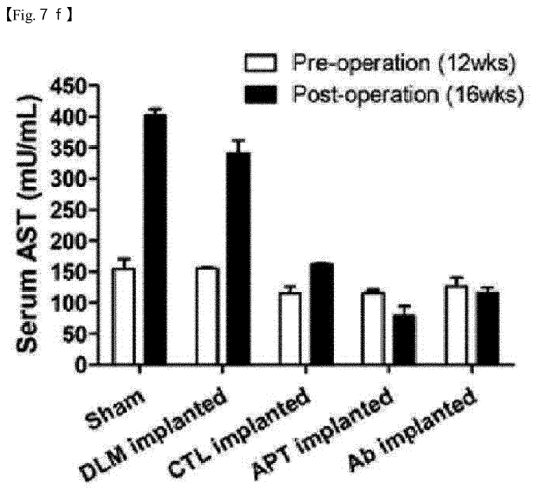

As illustrated in FIG. 7A, the liver-specific functionality in vivo was evaluated by transplanting a vascularized artificial liver into a rat model in which hepatic fibrosis was induced using thioacetamide (TAA). Eight weeks after TAA induction, rats received the xenograft transplantation of decellularized liver matrix (DLM transplant) or VBHL constructs (CTL-VBHL; CTL transplant, APT-VBHL; APT transplant, Ab-VBHL; Ab transplant).

Specific Experimental Groups are as Follows:
1) Sham
2) DLM implanted; implantation of cell-free decellularized scaffold alone
3) CTL implanted; implantation of artificial liver reconstructed without coating
4) APT implanted; implantation of artificial liver treated with anti-CD31 aptamer as coating agent
5) Ab implanted; implantation of artificial liver treated with anti-CD31 antibody as coating agent

Four weeks later, the livers of the recipient rats were harvested and subjected to H&E tissue staining and Picrosirius red tissue staining (red: collagen, green: cell cytoplasm).

As a result, as illustrated in FIGS. 7B and 7C, when the decellularized scaffold and the artificial liver were transplanted, the eosinophilic change and degree of fibrosis in the liver were decreased compared to the sham group. Among them, it was confirmed that the degree of liver fibrosis was reduced most when an artificial liver treated with the aptamer was transplanted.

In addition, as illustrated in FIG. 7D, it was confirmed that when the mRNA expressed in the liver of the recipient was analyzed, the expression of fibrosis-related markers (α-smooth muscle actin(Sma), Vimentin, Tgf-beta1, and Timp1) was clearly decreased in the APT implanted group.

Furthermore, as illustrated in FIGS. 7E and 7F, it was confirmed that when the ALT and AST levels, which are indicators of liver injury, were confirmed in the serum levels of rats. The serum ALT and AST levels after surgery in the APT implanted group were maintained at normal levels (between the red lines).

Therefore, the aptamer-treated artificial liver of the present invention demonstrates that enhanced liver-specific functionality is maintained in vivo, which assists the recipient's impaired liver function.

As described above, the present inventors confirmed for the first time that aptamers can be applied to artificial organs.

Based on the above results, when an aptamer is used as a coating agent for decellularized scaffolds, vasculature can be reconstructed more efficiently than anti-CD31 antibodies used in existing studies by enhancing blood vessel adhesion ability, viability, angiogenesis potential, and the like through the integrin-Akt signaling system. Therefore, a vascularized artificial liver produced using the present invention not only reduces thrombus formation in vivo, but also exhibits the effect of enhancing liver functionality. Further, this is a result that suggests the applicability of the artificial liver as a therapeutic agent for liver disease in terms of tissue engineering.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

When the aptamer of the present invention is used as a coating agent of a decellularized scaffold, it is possible to more efficiently reconstruct vasculature than existing antibodies by enhancing blood vessel adhesion ability, viability, angiogenesis potential, and the like. Therefore, a vascularized artificial liver produced using the aptamers exhibits the effects of reducing thrombogenesis in vivo as well as enhancing liver function, and thus is expected to be applied as a method for treating diseases by using artificial organs produced using the aptamers according to the present invention, so that there is industrial applicability.

1-20. (canceled)

21. A method for producing artificial organs, the method comprising a step of treating with aptamers.

22. The method of claim 21, wherein the method further comprises the following steps:
a) providing an organ from a tissue source;
b) decellularizing the provided organ; and
c) treating the decellularized organ with aptamers.

23. The method of claim 22, wherein the method further comprises one or more steps selected from the group consisting of the following steps:
d-1) recellularizing a decellularized organ into vascular endothelial cells;
d-2) recellularizing a decellularized organ into parenchymal cells; and
d-3) recellularizing a decellularized organ into non-parenchymal cells.

24. The method of claim 23, wherein the cells comprise stem cell-derived differentiated cells.

25. The method of claim 24, wherein the stem cells are one or more selected from the group consisting of induced pluripotent stem cells (iPSCs), embryonic stem cells, mesenchymal stromal cells (MSCs), off-the-shelf universal stem cells (USCs), marrow-derived stem cells, adipose tissue-derived stem cells and placenta-derived stem cells.

26. The method of claim 25, wherein the off-the-shelf universal stem cells have one or more of the following characteristics:
human leukocyte antigen (HLA) gene has been removed; and
“Don't eat me” signals are overexpressed.

27. The method of claim 21, wherein the aptamer is an anti-CD31 aptamer.

28. The method of claim 21, wherein the aptamer comprises a base sequence represented by SEQ ID NO: 1.

29. The method of claim 21, wherein the aptamer increases the expression of one or more selected from the group consisting of integrin beta 3 and phosphorylated Akt.

30. The method of claim 21, wherein the aptamer decreases the expression of cleaved caspase-3.

31. A composition for producing artificial organs, comprising aptamers.

32. A composition for coating blood vessels, comprising aptamers.


SEQUENCE LISTING (41 sequences)

SEQ ID NO: 1 - DNA, 40 bp, Artificial Sequence
  MISC_FEATURE (2)..(40): anti-CD31 aptamer, wherein n at position 2-40 is NapdU [5-(N-Napthylcarboxyamide)-2′-deoxyuridine]
gnagaggaggnacgnaangncngggnanaccccganaann

SEQ ID NO: 2 - DNA, 22 bp, Artificial Sequence
  human GAPDH_F
tgatgacatcaagaaggtggtg

SEQ ID NO: 3 - DNA, 21 bp, Artificial Sequence
  human GAPDH_R
accctgttgctgtagccaaat

SEQ ID NO: 4 - DNA, 21 bp, Artificial Sequence
  human ITGB1_F
cgtagcaaaggaacagcagag

SEQ ID NO: 5 - DNA, 25 bp, Artificial Sequence
  human ITGB1_R
ggtagtagaggtcaatgggatagtc

SEQ ID NO: 6 - DNA, 20 bp, Artificial Sequence
  human ITGB3_F
cctcatcaccatccacgacc

SEQ ID NO: 7 - DNA, 21 bp, Artificial Sequence
  human ITGB3_R
gttgttggctgtgtcccattt

SEQ ID NO: 8 - DNA, 22 bp, Artificial Sequence
  human VE-cadherin_F
cttcacccagaccaagtacaca

SEQ ID NO: 9 - DNA, 20 bp, Artificial Sequence
  human VE-cadherin_R
aatggtgaaagcgtcctggt

SEQ ID NO: 10 - DNA, 20 bp, Artificial Sequence
  human CLDN5_F
aagtgtacgactcggtgctg

SEQ ID NO: 11 - DNA, 20 bp, Artificial Sequence
  human CLDN5_R
aaacaggtagagcacgcctc

SEQ ID NO: 12 - DNA, 23 bp, Artificial Sequence
  human NOS3_F
aaagacaaggcagcagtggaaat

SEQ ID NO: 13 - DNA, 22 bp, Artificial Sequence
  human NOS3_R
tccacgatggtgactttggcta

SEQ ID NO: 14 - DNA, 18 bp, Artificial Sequence
  human CD63_F
cagctagagagccccgga

SEQ ID NO: 15 - DNA, 20 bp, Artificial Sequence
  human CD63_R
ttcattcctccttccaccgc

SEQ ID NO: 16 - DNA, 24 bp, Artificial Sequence
  human PLSCR1_F
ggcagccagagaactgttttaatc

SEQ ID NO: 17 - DNA, 20 bp, Artificial Sequence
  human PLSCR1_R
ggcaagtttgtttccgggtg

SEQ ID NO: 18 - DNA, 17 bp, Artificial Sequence
  human TBXAS1_F
tgcagagcacggttccc

SEQ ID NO: 19 - DNA, 22 bp, Artificial Sequence
  human TBXAS1_R
gtggagtaccatttcaggaggg

SEQ ID NO: 20 - DNA, 20 bp, Artificial Sequence
  human THBS1_F
agtcgtctctgcaacaaccc

SEQ ID NO: 21 - DNA, 22 bp, Artificial Sequence
  human THBS1_R
acaggcatccatcaattggaca

SEQ ID NO: 22 - DNA, 21 bp, Artificial Sequence
  human ALB_F
cgctattagttcgttacacca

SEQ ID NO: 23 - DNA, 20 bp, Artificial Sequence
  human ALB_R
tttacaacatttgctgccca

SEQ ID NO: 24 - DNA, 20 bp, Artificial Sequence
  human CYP1A2_F
cggacagcacttccctgaga

SEQ ID NO: 25 - DNA, 20 bp, Artificial Sequence
  human CYP1A2_R
aggcaggtagcgaaggatgg

SEQ ID NO: 26 - DNA, 20 bp, Artificial Sequence
  rat Gapdh_F
accacagtccatgccatcac

SEQ ID NO: 27 - DNA, 20 bp, Artificial Sequence
  rat Gapdh_R
tccaccaccctgttgctgta

SEQ ID NO: 28 - DNA, 19 bp, Artificial Sequence
  rat Cd63_F
catcaagaagcgtcgggga

SEQ ID NO: 29 - DNA, 20 bp, Artificial Sequence
  rat Cd63_R
acctgaactgctacgccaat

SEQ ID NO: 30 - DNA, 20 bp, Artificial Sequence
  rat Plscr1_F
ctgcgaggctttagggagag

SEQ ID NO: 31 - DNA, 20 bp, Artificial Sequence
  rat Plscr1_R
ctctgaggtctgcaaggtgg

SEQ ID NO: 32 - DNA, 20 bp, Artificial Sequence
  rat Thbs1_F
tagctggaaatgtggtgcgt

SEQ ID NO: 33 - DNA, 20 bp, Artificial Sequence
  rat Thbs1_R
agcaagcatcaggcacttct

SEQ ID NO: 34 - DNA, 20 bp, Artificial Sequence
  rat alpha-Sma_F
catcaccaactgggacgaca

SEQ ID NO: 35 - DNA, 20 bp, Artificial Sequence
  rat alpha-Sma_R
tccgttagcaaggtcggatg

SEQ ID NO: 36 - DNA, 20 bp, Artificial Sequence
  rat Vimentin_F
cactcacctgcgaagtggat

SEQ ID NO: 37 - DNA, 20 bp, Artificial Sequence
  rat Vimentin_R
tggtattcacgaaggtggcg

SEQ ID NO: 38 - DNA, 20 bp, Artificial Sequence
  rat Tgf-beta1_F
ctgctgacccccactgatac

SEQ ID NO: 39 - DNA, 20 bp, Artificial Sequence
  rat Tgf-beta1_R
agccctgtattccgtctcct

SEQ ID NO: 40 - DNA, 20 bp, Artificial Sequence
  rat Timp1_F
tgctcaaaggattcgacgct

SEQ ID NO: 41 - DNA, 20 bp, Artificial Sequence
  rat Timp1_R
agcagggctcagattatgcc